(19) United States
(12) Patent Application Publication
(10) Pub. No.: US 2005/0032742 A1
(43) Pub. Date: Feb. 10, 2005

(54) Chemo-enzymatic synthesis of sialylated oligosaccharides

(76) Inventors: Shawn DeFrees, North Wales, PA (US); Edward J McGuire, Furlong, PA (US)

Correspondence Address: MORGAN, LEWIS & BOCKIUS LLP (SF), 2 PALO ALTO SQUARE, PALO ALTO, CA, 94306, US

(21) Appl. No.: 10/485,892

(22) PCT Filed: Aug. 1, 2002
(86) PCT No.: PCT/US02/24574

(30) Foreign Application Priority Data
Aug. 17, 2001 (US) 60313278
Jan. 23, 2002 (US) 60351444

(51) Int. Cl. A61K 31/739; C08B 37/00; C12P 19/26

(52) U.S. Cl. 514/54; 435/84; 536/53

(57) ABSTRACT

In vitro/cell-free process of preparing a sialylated oligosaccharides are described. The sialylated oligosaccharides include gangliosides. The oligosaccharides linked to various moieties including sphingoids and ceramides. Novel compounds that comprise sphingoid groups are disclosed. The compounds include sialylated oligosaccharides including gangliosides as well as various sphingoids and ceramides.

### BACKGROUND OF THE INVENTION

1. Field of the Invention

This invention pertains to the field of methods for preparing oligosaccharides that include one or more sialyl groups.

2. Background

Gangliosides are a class of glycosphingolipids that have a structure containing a carbohydrate moiety linked to a ceramide. The carbohydrate moiety includes at least one monosaccharide and a sialic acid moiety. The sialic acid moiety is composed of one or more sialic acid groups (N-acetyl or N-glycolyl neuraminic acid).

Gangliosides are classified according to the number of monosaccharides in the sugar moiety and the number of sialic acid groups present in the structure. Gangliosides are known as mono-, di-, tri- or poly-sialogangliosides, depending upon the number of sialic acid residues. Abbreviations employed to identify these molecules include “GM1”, “GD3”, “GT1”, etc., with the “G” standing for ganglioside, “M”, “D” or “T”, etc. referring to the number of sialic acid residues, and the number or number plus letter (e.g., “GT1a”), referring to the elution order in a TLC assay observed for the molecule. See, Lehninger, Biochemistry, pg. 294-296 (Worth Publishers, 1981); Wiegandt, Glycolipids: New Comprehensive Biochemistry (Neuberger et al., ed., Elsevier, 1985), pp. 199-260.

For example, the international symbol GM₁ₐ designates one of the more common gangliosides, which has been extensively studied. The “M” in the symbol indicates that the ganglioside is a monosialoganglioside and “1” defines its position in a TLC elution profile. The subscripts “a”, “b” or “c” also indicate the positions in a TLC assay of the particular ganglioside. The terminal saccharide is the saccharide, which is located at the end of the carbohydrate moiety, which is opposite to the end that is attached to the ceramide moiety.

Hundreds of glycosphingolipids (GSLs) are derived from glucosylceramide (GlcCer), which is enzymatically formed from ceramide and UDP-glucose. The enzyme involved in GlcCer formation is UDP-glucose:N-acylsphingosine glucosyltransferase (GlcCer synthase). The rate of GlcCer formation under physiological conditions may depend on the tissue level of UDP-glucose, which in turn depends on the level of glucose in a particular tissue (Zador, I. Z. et al., J. Clin. Invest. 91: 797-803 (1993)). In vitro assays based on endogenous ceramide yield lower synthetic rates than mixtures containing added ceramide, suggesting that tissue levels of ceramide are also normally rate-limiting (Brenkert, A. et al., Brain Res. 36: 183-193 (1972)).

The level of GSLs controls a variety of cell functions, such as growth, differentiation, adhesion between cells or between cells and matrix proteins, binding of microorganisms and viruses to cells, and metastasis of tumor cells. In addition, the GlcCer precursor, ceramide, may cause differentiation or inhibition of cell growth (Bielawska, A. et al., FEBS Letters 307: 211-214 (1992)) and be involved in the functioning of vitamin D₃, tumor necrosis factor-α, interleukins, and apoptosis (programmed cell death). The sphingols (sphingoid bases), precursors of ceramide, and products of ceramide catabolism, have also been shown to influence many cell systems, possibly by inhibiting protein kinase C (PKC).

Gangliosides are known to be functionally important in the nervous system and it has been claimed that gangliosides are useful in the therapy of peripheral nervous system disorders. Numerous gangliosides and derivatives thereof have been used to treat a wide variety of nervous system disorders including Parkinson's disease (Ganglioside GM₁ is currently being used in phase II clinical development for the treatment of Parkinson's Disease (FIDIA, Italy)), and cerebral ischemic strokes (see, U.S. Pat. Nos. 4,940,694; 4,937,232; and 4,716,223). Gangliosides have also been used to affect the activity of phagocytes (U.S. Pat. No. 4,831,021) and to treat gastrointestinal disease-producing organisms (U.S. Pat. No. 4,762,822). The gangliosides GM₂ and GD_{2,} purified from animal brain, have been conjugated to keyhole limpet hemacyanin (KLH) and mixed with adjuvant QS21, and used to elicit immune responses to these gangliosides, as the basis of a cancer vaccine in phase II and III trials (Progenics, Tarrytown, N.Y.). Ganglioside GM₃ is being investigated for use as an anti-cancer agent (WO 98/52577; Nole et al., Exp. Neurology 168: 300-9 (2001)). )). Glycolipids are also of interest in the treatment of inflammatory bowel disease. See, Tubaro et al., Naunyx-Schmiedebergg's Arch. Pharmacol. 348: 670-678 (1993).

Gangliosides are generally isolated via purification from tissue, particularly from animal brain (GLYCOLIPID METHODOLOGY, Lloyd A. Witting Ed., American Oil Chemists Society, Champaign, III. 187-214 (1976); U.S. Pat. Nos. 5,844,104; 5,532,141; Sonnino et al., J. Lipid Res. 33: 1221-1226 (1992); Sonnino et al., Ind. J. Biochem. Biophys., 25: 144-149 (1988); Svennerholm, Adv. Exp. Med. Biol. 125: 533-44 (1980)). Gangliosides have been isolated from bovine buttermilk (Ren et al., J. Bio. Chem. 267: 12632-12638 (1992); Takamizawa et al., J. Bio. Chem. 261: 5625-5630(1986)). Even under optimum conditions, the yields of pure gangliosides, e.g., GM2 and GM3, are vanishingly small. Moreover, purification from mammalian tissue carries with it the risk of transmitting contaminants such as viruses, prion particles, and so forth. Alternate methodologies for securing ganglioside specific antibodies are thus highly desirable.

Due to the importance of gangliosides, efforts have been expended to develop methods of synthesizing pure gangliosides in high yields. Methods of chemically synthesizing gangliosides are described in Hasegawa et al., J. Carbohydrate Chemistry, 11(6): 699-714 (1992) and Sugimoto et al., Carbohydrate Research, 156: C1-C5 (1986). U.S. Pat. No. 4,918,170 discloses the synthesis of GM3 and GM4. Schmidt et al. describe the chemical synthesis of GM3 (U.S. Pat. No. 5,977,329). The references describe multi-step synthetic procedures using laborious protection-activation-coupling-deprotection strategies, at each step of which the intermediate is purified, generally by a combination of extraction and column chromatography. Moreover, none of the synthetic methods is appropriate for the large-scale preparation of gangliosides.

In view of the difficulties associated with the chemical synthesis of carbohydrates, the use of enzymes to synthesize the carbohydrate portions of glycoproteins is a promising approach to preparing glycoproteins. Enzyme-based syntheses have the advantages of regioselectivity and stereoselectivity. Moreover, enzymatic syntheses can be performed using unprotected substrates. Three principal classes of enzymes are used in the synthesis of carbohydrates, glycosyltransferases (e.g., sialyltransferases, oligosaccharyltransferases, N-acetylglucosaminyltransferases), Glycoaminidases (e.g., PNGase F) and Glycosidases. The glycosidases are further classified as exoglycosidases (e.g., β-mannosidase, β-glucosidase), and endoglycosidases (e.g., Endo-A, Endo-M). Each of these classes of enzymes has been successfully used to prepare carbohydrates. For a general review, see, Crout et al., Curr. Opin. Chem. Biol. 2: 98-111 (1998) and Arsequell, supra.

Glycosyltransferases have been used to prepare oligosaccharides, and have been shown to be very effective for producing specific products with good stereochemical and regiochemical control. For example, β-1,4-galactosyltransferase was used to synthesize lactosamine, illustrating the utility of glycosyltransferases in the synthesis of carbohydrates (see, e.g., Wong et al., J. Org. Chem. 47: 5416-5418 (1982)). Moreover, numerous synthetic procedures have made use of α-sialyltransferases to transfer sialic acid from cytidine-5′-monophospho-N-acetylneuraminic acid to the 3-OH or 6-OH of galactose (see, e.g., Kevin et al., Chem. Eur. J. 2: 1359-1362 (1996)). For a discussion of recent advances in glycoconjugate synthesis f6r therapeutic use, see, Koeller et al., Nature Biotechnology 18: 835-841 (2000).

Glycosidases normally catalyze the hydrolysis of a glycosidic bond, however, under appropriate conditions they can be used to form this linkage. Most glycosidases used for carbohydrate synthesis are exoglycosidases; the glycosyl transfer occurs at the non-reducing terminus of the substrate. The glycosidase takes up a glycosyl donor in a glycosyl-enzyme intermediate that is either intercepted by water to give the hydrolysis product, or by an acceptor, to give a new glycoside or oligosaccharide. An exemplary pathway using a exoglycoside is the synthesis of the core trisaccharide of all N-linked glycoproteins, including the notoriously difficult β-mannoside linkage, which was formed by the action of β-mannosidase (Singh et al., Chem. Commun. 993-994 (1996)).

Although their use is less common than that of the exoglycosidases, endoglycosidases have also been utilized to prepare carbohydrates. Methods based on the use of endoglycosidases have the advantage that an oligosaccharide, rather than a monosaccharide, is transferred. Oligosaccharride fragments have been added to substrates using endo-β-N-acetylglucosamines such as endo-F, endo-M (Wang et al., Tetrahedron Lett. 37: 1975-1978); and Haneda et al., Carbohydr. Res. 292: 61-70 (1996)).

Methods combining both chemical and enzymatic synthetic elements are also known. For example, Yamamoto and coworkers (Carbohydr. Res. 305: 415-422 (1998)) reported the chemoenzymatic synthesis of the substrate, glycosylated Peptide T, using an endoglyosidase. The N-acetylglucosaminyl peptide was synthesized by purely chemical means. The peptide was subsequently enzymatically elaborated with the oligosaccharide of human transferrin substrate. The saccharide portion was added to the peptide by treating it with an endo-β-N-acetylglucosaminidase. The resulting glycosylated peptide was highly stable and resistant to proteolysis when compared to the peptide T and N-acetylglucosaminyl peptide T.

Despite the many advantages of the enzymatic synthesis methods set forth above, in some cases, deficiencies remain. Since the biological activity of many commercially important recombinantly and transgenically produced substrates depends upon the presence of a particular glycoform, or the absence of a particular glycoform, a need exists for an in vitro procedure to enzymatically modify glycosylation patterns, particularly on substrates such as ceramide, sphingosine and their analogues. The present invention fulfills these and other needs.

### SUMMARY OF THE INVENTION

It has now been discovered that gangliosides and ganglioside analogues are readily synthesized in excellent yields, in high purity and with exquisite stereochemical specificity using an enzymatic synthesis protocol. Thus, in response to the need for improved methods of preparing glycosylated species, the present invention provides methods for the enzyme-mediated formation of conjugates between glycosyl groups and selected substrates.

In a first aspect the present invention provides a method of glycosylating a species according to Formula I:
(saccharide)_{q}-X (I).

The method includes contacting (saccharide)ₛ-X with a trans-sialidase or glycosyltransferase in presence of appropriate donor to yield (saccharide)ₛ₊₁-X. The product of the first reaction is optionally contacted with a trans-sialidase or glycosyltransferase in presence of appropriate donor to yield (saccharide)ₛ₊₂-X. The product of the second reaction is optionally contacted with a trans-sialidase or glycosyltransferase in presence of appropriate donor to yield (saccharide)ₛ₊₃-X. The process continues until the desired saccharide structure is built up. In the structures provided above, s is an integer from 0 to about 30. The symbol q represents an integer from 2 to about 30. It is generally preferred that the process of the invention include at least one sialylation that is mediated by a trans-sialidase, and two glycosylations that are mediated by the action of one or more glycosyltransferases. The method also preferably is practiced in the absence of a cellular component to the reaction mixture, and is preferably performed entirely in vitro.

In another aspect, the invention provides methods for glycosylating ceramide, sphingosine and their analogues.

In yet a further aspect, the invention provides ceramide and sphingosine derivatives in which the alkyl chain of the sphingosine backbone includes two or more degrees of unsaturation. Also provides are pharmaceutical compositions that include the ceramide and sphingosine derivatives of the invention.

Additional objects and advantages of the present invention will be apparent from the detailed description that follows.

### BRIEF DESCRIPTION OF THE DRAWINGS

FIG. 1 is Scheme 1, Pathway 1, showing an overview of the GM and GD series syntheses beginning from an aglycone and tracing the sequential addition of saccharide units.

FIG. 2 is Scheme 1, Pathway 2 showing the synthesis of the GM and GD series beginning from a sphingoid and tracing the sequential addition of saccharide units.

FIG. 3 is Scheme 2, showing the synthesis of GM₁(d18:2) from glucosyl-sphingosine d18:2. Scheme 2 outlines a general strategy by which a glucosyl-sphingosine (1) is converted to a lactosyl sphingosine (2) by a galactosyltransferase reaction. Lactosyl sphingosine (2) is converted to lyso-GM₃ (3) by a trans-sialidase reaction. The lyso-GM₃ (3) is acylated to create GM₃ (4). The ganglioside GM₃ (4) is further processed to add additional saccharide. GM₃ (4) is first converted to GM₂ (5) by a GalNAc transferase reaction and subsequently GM₂ (5) is converted to GM₁(6) by a galactosyltransferase reaction.

FIG. 4 is Scheme 3, showing the synthesis of GM₃(d18:2) from glucosyl-sphingosine d18:2. Scheme 3 depicts a general strategy by which GM₃ is made from a glucosyl-sphingosine. The glucosyl-sphingosine is converted to a lactosyl sphingosine by a galactosyltransferase reaction. Lactosyl sphingosine is converted to lactosyl ceramide by an acylation reaction. The lactosyl ceramide is converted to GM₃ by a trans-sialidase reaction.

FIG. 5 is Scheme 4, showing the synthesis of GD₃(d18:2), GD₂(d18:2), or GD_{1b}(d18:2) from lyso-GM3(d18:2). Scheme 4 outlines a general strategy by which gangliosides in the GD series are made by acylation of reaction products from the addition of saccharides to lyso-GM₃. Lyso-GM₃ (3) is converted to Lyso-GD₃ (8) by a sialyltransferase reaction. Lyso-GD₃ (8) can be converted to GD₃ (9) by acylation or can serve as an acceptor for a saccharide addition such as its conversion to Lyso-GD₂ (10) by a GalNAc transferase reaction. Similarly, Lyso-GD₂ (10) can be converted to GD₂ (11) by acylation or can serve as an acceptor for a saccharide addition such as its conversion to Lyso-GD₁ (12) by a Galactosyltransferase reaction. Lyso-GD₁ (12) can be converted to GD₁ (14) by acylation.

FIG. 6 is Scheme 5, showing the synthesis of GM₁(d18:1), GM₂(d18:1), GM₁(d18:1), or fucosyl-GM₁(d18:1) from sphingosine d18:1. Scheme 5outlines a general strategy by which gangliosides in the GM series can be made by acylation of reaction products produced by adding saccharides to a sphingosine free of fatty acid.

FIG. 7 is Scheme 6, showing the synthesis of GD₃(d18:1), GD₂(d18:1), GD_{1b}(d18:1), or GT_{1b} from lyso-GM₃(d18:1). According to this general strategy, the GD series members are created by acylation of their lyso-GD forms rather than through addition of saccharides to acylated members.

FIG. 8 displays representative examples of ceramides (where R₁═H) and sphingosines (where R₁=fatty acid or fatty acid derivative) as aglycones. Exemplary compounds prepared by a method of the invention include those in which the saccharide is absent, or an oligosaccharide with 2-20 members.

FIG. 9 is Scheme 8, showing the synthesis of representative poly-sialylated sphingosine and ceramide molecules. Scheme 8 shows an example of a general strategy for polymeric addition of sialic acid by sialyltransferase reaction to non-acylated sphingoids.

FIG. 10 is Scheme 9, showing the synthesis of GD gangliosides, as well as poly-sialylated GD₃, from GM₃(d18:1). Scheme 9 depicts an example of a general strategy for addition of repeating sialic acid monomers.

FIG. 11 shows exemplary compounds of the formula oligosaccharide-X, prepared by methods of the invention.

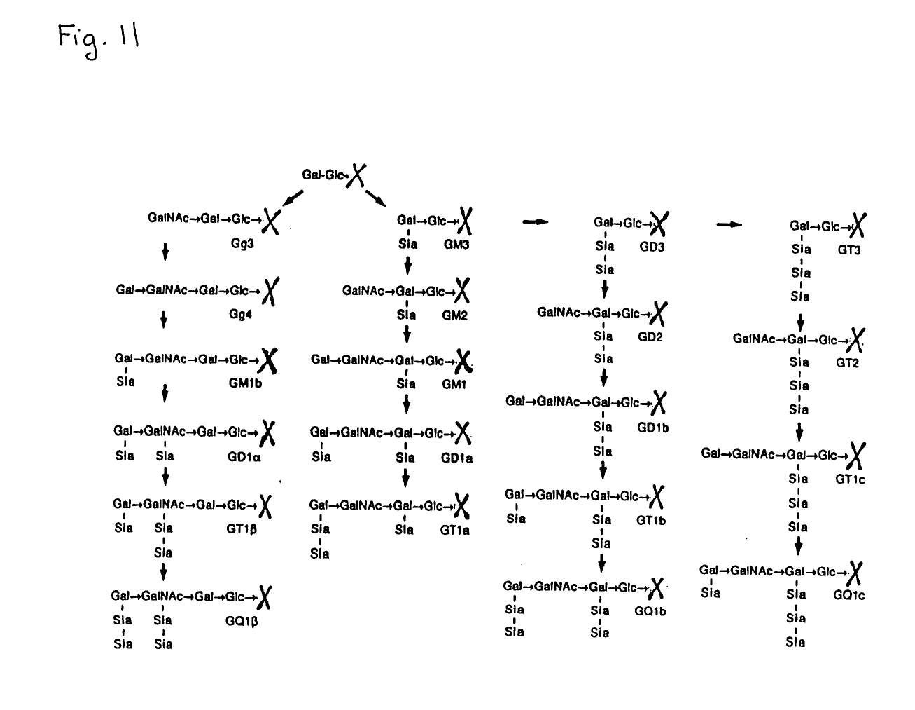

### DETAILED DESCRIPTION OF THE INVENTION AND THE PREFERRED EMBODIMENTS

Abbreviations

Abrreviations of saccharide moieties refer to both substituted and unsubstituted analogues of the saccharides. Thus, arabinosyl; Fru, fructosyl; Fuc, fucosyl; Gal, galactosyl; GalNAc, N-acetylgalactosyl; Glc, glucosyl; GlcNAc, N-acetylglucosyl; Man, mannosyl; ManAc, mannosyl acetate; Xyl, xylosyl; and Sia and NeuAc, sialyl (N-acetylneuraminyl). The abbreviations are intended to encompass both unmodified saccharyl moieties and substituted or other analogues thereof.

Definitions

Unless defined otherwise, all technical and scientific terms used herein generally have the same meaning as commonly understood by one of ordinary skill in the art to which this invention belongs. Generally, the nomenclature used herein and the laboratory procedures in molecular biology, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those known and employed in the art. Standard techniques, or modifications thereof, are used for chemical syntheses and chemical analyses.

“Analyte”, as used herein, means any compound or molecule of interest for which a diagnostic test is performed, such as a biopolymer or a small molecular bioactive material. An analyte can be, for example, a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc., without limitation.

“Peptide” refers to a polymer in which the monomers are amino acids and are joined together through amide bonds, alternatively referred to as a polypeptide. When the amino acids are a-amino acids, either the L-optical isomer or the D-optical isomer can be used. Additionally, unnatural amino acids, for example, β-alanine, phenylglycine and homoarginine are also included. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Modifications can also include 3′ and 5′ modifications such as capping with a PL, a fluorophore or another moiety.

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

An “acceptor moiety” for a glycosyltransferase is an oligosaccharide structure that can act as an acceptor for a particular glycosyltransferase. When the acceptor moiety is contacted with the corresponding glycosyltransferase and sugar donor moiety, and other necessary reaction mixture components, and the reaction mixture is incubated for a sufficient period of time, the glycosyltransferase transfers sugar residues from the sugar donor moiety to the acceptor moiety. The acceptor moiety will often vary for different types of a particular glycosyltransferase. For example, the acceptor moiety for a mammalian galactoside 2-L-fucosyltransferase (α1,2-fucosyltransferase) will include a Galβ1,4-GlcNAc-R at a non-reducing terminus of an oligosaccharide; this fucosyltransferase attaches a fucose residue to the Gal via an α1,2 linkage. Terminal Galβ1,4-GlcNAc-R and Galβ1,3-GlcNAc-R are acceptor moieties for α1,3 and α1,4-fucosyltransferases, respectively. These enzymes, however, attach the fucose to the GlcNAc residue of the acceptor. Accordingly, the term “acceptor moiety” is taken in context with the particular glycosyltransferase of interest for a particular application. Acceptor moieties for additional fucosyltransferases, and for other glycosyltransferases, are described herein.

The term “sialic acid” refers to any member of a family of nine-carbon carboxylated sugars. Also included are sialic acid analogues that are derivatized with linkers, reactive functional groups, detectable labels and targeting moieties. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids: Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques. A “recombinant polypeptide” is one that has been produced by a recombinant cell.

The term “isolated” refers to a material that is substantially or essentially free from components, which are used to produce the material. For compositions produced by a method of the invention, the term “isolated” refers to material that is substantially or essentially free from components, which normally accompany the material in the mixture used to prepare the composition. “Isolated” and “pure” are used interchangeably. Typically, isolated compounds produced by the method of the invention have a level of purity preferably expressed as a range. The lower end of the range of purity for the peptide compounds is about 60%, about 70% or about 80% and the upper end of the range of purity is about 70%, about 80%, about 90% or more than about 90%.

When the compounds produced be a method of the invention are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, or a similar means).

“Essentially each member of the population,” as used herein, describes a characteristic of a population of compounds produced by a method of the invention in which a selected percentage of the glycosyl donor added to a precursor substrate are added to identical acceptor sites on the individual members of a population of substrate. “Essentially each member of the population” speaks to the “homogeneity” of the sites on the substrate that are conjugated to a glycosyl donor and refers to compounds of the invention, which are at least about 80%, preferably at least about 90% and more preferably at least about 95% homogenous.

“Homogeneity,” refers to the structural consistency across a population of acceptor moieties to which the glycosyl donors are conjugated. Thus, if at the end of a glycosylation reaction, each glycosyl donor transferred during the reaction is conjugated to an acceptor site having the same structure, the composition is said to be about 100% homogeneous. Homogeneity is typically expressed as a range. The lower end of the range of homogeneity for the peptide conjugates is about 60%, about 70% or about 80% and the upper end of the range of purity is about 70%, about 80%, about 90% or more than about 90%.

When the compositions prepared by a method of the invention are more than or equal to about 90% homogeneous, their homogeneity is also preferably expressed as a range. The lower end of the range of homogeneity is about 90%, about 92%, about 94%, about 96% or about 98%. The upper end of the range of purity is about 92%, about 94%, about 96%, about 98% or about 100% homogeneity. The purity of the peptide conjugates is typically determined by one or more methods known to those of skill in the art, e.g., liquid chromatography-mass spectrometry (LC-MS), matrix assisted laser desorption mass time of flight spectrometry (MALDITOF), capillary electrophoresis, and the like.

“Substantially uniform glycoform” or a “substantially uniform glycosylation pattern,” when referring to a composition prepared by a method of the invention, refers to the percentage of acceptor moieties that are glycosylated by the trans-sialidase or glycosyltransferase of interest (e.g., fucosyltransferase). For example, in the case of a α1,2 fucosyltransferase, a substantially uniform fucosylation pattern exists if substantially all (as defined below) of the Galβ1,4-GlcNAc-R and sialylated analogues thereof are fucosylated in a composition prepared by a method of the invention. It will be understood by one of skill in the art, that the starting material may contain glycosylated acceptor moieties (e.g., fucosylated Galβ1,4-GlcNAc-R moieties). Thus, the calculated percent glycosylation will include acceptor moieties that are glycosylated by the methods of the invention, as well as those acceptor moieties already glycosylated in the starting material.

The term “substantially” in the above definitions of “substantially uniform” generally means at least about 40%, at least about 70%, at least about 80%, or more preferably at least about 90%, and still more preferably at least about 95% of the acceptor moieties for a particular glycosyltransferase are glycosylated.

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

All oligosaccharides described herein are described with the name or abbreviation for the non-reducing saccharide (i.e., Gal), followed by the configuration of the glycosidic bond (α or β), the ring bond (1 or 2), the ring position of the reducing saccharide involved in the bond (2, 3, 4, 6 or 8), and then the name or abbreviation of the reducing saccharide (i.e., GlcNAc). Each saccharide is preferably a pyranose. For a review of standard glycobiology nomenclature see, Essentials of Glycobiology Varki et al. eds. CSHL Press (1999).

As used herein, “linking member” refers to a covalent chemical bond that includes at least one heteroatom. Exemplary linking members include —C(O)NH—, —C(O)O—, —NH—, —S—, —O—, and the like.

The term “targeting moiety,” as used herein, refers to species that will selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. Exemplary targeting moieties include antibodies, antibody fragments, transferrin, HS-glycoprotein, coagulation factors, serum proteins, β-glycoprotein, G-CSF, GM-CSF, M-CSF, EPO, saccharides, lectins, receptors, ligand for receptors, proteins such as BSA and the like. The targeting group can also be a small molecule, a term that is intended to include both non-peptides and peptides.

The symbol [symbol] , whether utilized as a bond or displayed perpendicular to a bond indicates the point at which the displayed moiety is attached to the remainder of the molecule, solid support, etc.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl,” and “alkylene.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetaahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymnethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)S—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

Introduction

The biological activity of many compounds, e.g, glycolipids, depends upon the presence or absence of a particular glycoform. Advantages of glycolipid compositions that have altered glycosylation patterns include, for example, increased therapeutic half-life of due to reduced clearance rate, enhanced bioavailability, and altered bioactivity. Moreover, altering the glycosylation pattern of a compound can mask antigenic determinants, thus reducing or eliminating an immune response against the compound. Alteration of the glycoform of a glycolipid can also be used to target the glycolipid to a particular tissue or cell surface receptor that is specific for the altered oligosaccharide. The altered oligosaccharide can also be used as an inhibitor of the receptor, preventing binding of its natural ligand. The present invention provides enzymatic methods for preparing glycoysylated substrates. The methods of the invention are exemplified herein by reference to their application to the synthesis of glycolipids, such as ceramides, sphingosines and their analogues. The focus of the discussion is for clarity of illustration, and those of skill will appreciate that the invention is not limited to the preparation of glycolipids.

The Methods

The present invention provides methods of preparing species having a selected glycosylation pattern. The invention is broadly directed to the enzymatically mediated, cell-free, in vitro glycosylation of a substrate. As one of skill will understand, the invention can be practiced on substantially any substrate including, but not limited to, peptides, nucleic acids, synthetic polymers, small organic radicals, and components of lipids. The invention is exemplified herein by its application to the preparation of glycolipids, specifically gangliosides. The focus of the discussion on gangliosides is for clarity of illustration only and does not limit the scope of the invention.

Thus, in a first aspect, the present invention provides a method of glycosylating a species according to Formula I:
(saccharide)_{q}-X (I).
The method includes contacting (saccharide)ₛ-X with a trans-sialidase or glycosyltransferase in presence of appropriate donor to yield (saccharide)ₛ₊₁-X. The product of the first reaction is optionally contacted with a trans-sialidase or glycosyltransferase in presence of appropriate donor to yield (saccharide)ₛ₊₂-X. The product of the second reaction is optionally contacted with a trans-sialidase or glycosyltransferase in presence of appropriate donor to yield (saccharide)ₛ₊₃-X. The process continues until the desired saccharide structure is built up. In the structures provided above, s is an integer from 0 to about 30. The symbol q represents an integer from 2 to about 30. It is generally preferred that the process of the invention include at least one sialylation that is mediated by a trans-sialidase, and two glycosylations that are mediated by the action of one or more glycosyltransferases. The method also preferably is practiced in the absence of a cellular component to the reaction mixture, and is preferably performed entirely in vitro.

In an alternative embodiment, the first glycosylation step utilizes a sialyltransferase and a sialic acid donor, rather than a trans-sialidase.

In an exemplary embodiment, the terminus of the saccharide that is not attached to X is a galactose residue. If a galactose residue is not present one is optionally added by, for example, contacting the saccharide construct with a galactosyltransferase.

As will be appreciated by those of skill in the art, the individual glycosylation steps of the method of the invention are practiced in any order that provides the desired structure. The only practical limitation upon the arrangement of steps is that the substrate must include an acceptor for the glycosyl unit that is to be added at a particular step. The acceptor can be added to the substrate by the method of the invention or it can be present on the native substrate. In addition to its being appended to the substrate structure by one or more glycosylation reactions, the acceptor can be exposed by trimming back glycosyl units that mask the desired acceptor. Moreover, the substrate can be trimmed back to a moiety that is a suitable acceptor for a structure that is to become the acceptor for the desired glycosylation step. See, for example WO 98/31826.

Addition or removal of carbohydrate moieties present on the substrate is accomplished either chemically or enzymatically. Chemical deglycosylation is preferably brought about by exposure of the substrate to trifluoromethanesulfonic acid, or an equivalent compound. Chemical deglycosylation is described by Hakimuddin et al., Arch. Biochem. Biophys. 259: 52 (1987) and by Edge et al., Anal. Biochem. 118: 131 (1981). Enzymatic cleavage of carbohydrate moieties on a substrate can be achieved by the use of a variety of endo- and exo-glycosidases as described by Thotakura et al., Meth. Enzymol. 138: 350 (1987).

Chemical addition of glycosyl moieties is carried out by any art-recognized method. Enzymatic addition of sugar moieties is preferably achieved using the methods set forth herein. Other useful methods of adding sugar moieties are disclosed in U.S. Pat. Nos. 5,876,980, 6,030,815, 5,728,554, and 5,922,577.

In another exemplary embodiment, the invention provides an in vitro, cell-free, enzymatic method for preparing a compound according to Formula II: 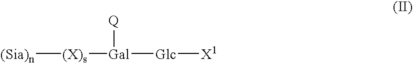
In Formula II, X¹ represents substituted or unsubstituted alkyl, a detectable label, carrier molecule or a targeting moiety. The symbol X represents a member selected from: 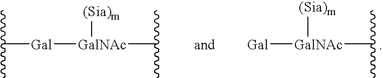
The symbol m represents an integer from 0 to 20. The symbol Q represents a member selected from: 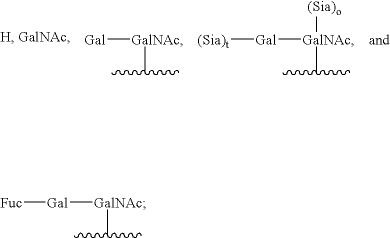
The symbols n, o and t represent integers independently selected from 0 to 20.

The method includes: (a) contacting with a trans-sialidase and a Sia donor, a substrate according to Formula III: 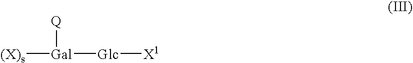
under conditions appropriate for the trans-sialidase to transfer a Sia moiety from the donor to the substrate, thereby forming a compound according to Formula II. The sialic acid moiety may optionally be transferred to the substrate by means of a sialyltransferase and a sialic acid donor.

Those of skill in the art will appreciate that the method of the invention may also commence upon a substrate having the structure: Glc-X¹, in which case, the first step is generally the addition of a Gal moiety using a galactosyltransferase and a galactose donor.

In another exemplary embodiment, the invention provides a method that further includes: (b) contacting the compound formed in step (a) with a GalNAc-transferase and a GalNAc donor under conditions appropriate for the GalNAc-transferase to transfer a GalNAc moiety from the donor to the compound formed in step (a).

In an alternative embodiment, the method includes: (b) contacting the compound formed in step (a) with a Sia-transferase and a Sia donor under conditions appropriate for the Sia-transferase to transfer a Sia moiety from the donor to the compound formed in step (a).

In a further exemplary embodiment, the method further includes: (c) contacting the compound formed in step (b) with a Gal-transferase and a Gal donor under conditions appropriate for the Gal-transferase to transfer a Gal moiety from the donor to the compound formed in step (b).

In an alternative embodiment, the method includes: (c) contacting the compound formed in step (b) with a GalNAc-transferase and a GalNAc donor under conditions appropriate for the GalNAc-transferase to transfer a GalNAc moiety from the donor to the compound formed in step (b).

In yet another exemplary embodiment, the method of the invention further includes: (c) contacting the compound formed in step (b) with a Sia-transferase and a Sia donor under conditions appropriate for the Sia-transferase to transfer a Sia moiety from the donor to the compound formed in step (b).

The method of the invention optionally includes: (d) contacting the compound formed in step (c) with a trans-sialidase and a Sia donor under conditions appropriate for the trans-sialidase to transfer a Sia moiety from the donor to the compound formed in step (c).

In a further exemplary embodiment, the method provides for: (d) contacting the compound formed in step (c) with a Fuc-transferase and a Fuc donor under conditions appropriate for the Fuc-transferase to transfer a Fuc moiety from the donor to the compound formed in step (c).

In an alternative embodiment, the method includes: (d) contacting the compound formed in step (c) with a Gal-transferase and a Gal donor under conditions appropriate for the Gal-transferase to transfer a Gal moiety from the donor to the compound formed in step (c).

In a further exemplary embodiment, the method includes: (d) contacting the compound formed in step (c) with a GalNAc-transferase and a GalNAc donor under conditions appropriate for the GalNAc-transferase to transfer a GalNAc moiety from the donor to the compound formed in step (c).

In yet another embodiment, the method further includes: (e) contacting the compound formed in step (d) with a Sia-transferase and a Sia donor under conditions appropriate for the Sia-transferase to transfer a Sia moiety from the donor to the compound formed in step (d).

In yet a further exemplary embodiment, the method further includes: (e) contacting the compound formed in step (d) with a trans-sialidase and a Sia donor under conditions appropriate for the trans-sialidase to transfer a Sia moiety from the donor to the compound formed in step (d).

In an alternative embodiment, the method includes: (e) contacting the compound formed in step (d) with a Gal-transferase and a Gal donor under conditions appropriate for the Gal-transferase to transfer a Gal moiety from the donor to the compound formed in step (d).

In another exemplary embodiment, the method provides for: (f) contacting the compound formed in step (e) with a Sia-transferase and a Sia donor under conditions appropriate for the Sia-transferase to transfer a Sia moiety from the donor to the compound formed in step (e).

In a further embodiment, the method includes: (f) contacting the compound formed in step (e) with a trans-sialidase and a Sia donor under conditions appropriate for the trans-sialidase to transfer a Sia moiety from the donor to the compound formed in step (e).

Those of skill will appreciate that a step utilizing a trans-sialidase can be replaced by a step using a sialyltransferase. Moreover, a trans-sialidase-mediated addition of sialic acid may be preceded by a sialic acid transfer mediated by a sialyltransferase.

In another embodiment, the method includes: (g) prior to step (a), contacting a substrate according to Formula IV:
Q-Gal-Glc-X¹ (IV)
with a GalNAc-transferase and a GalNAc donor under conditions appropriate for said GalNAc-transferase to transfer a GalNAc moiety from said donor to said substrate. The identity of Q and X¹ are as described for Formula II.

In a still further exemplary embodiment, the method includes: (h) contacting the compound formed in step (g) with a Gal-transferase and a Gal donor under conditions appropriate for the Gal-transferase to transfer a Gal moiety from the donor to the compound formed in step (g).

In another embodiment, the method includes: (i) following step (a), contacting the compound formed in step (a) with a Sia-transferase and a Sia donor under conditions appropriate for the Sia-transferase to transfer a Sia moiety from the donor to the compound formed in step (a).

The method also provides for: (j) repeating step (i) a selected number of times, thereby forming a poly(sialic acid) substituent on the compound.

In an additional exemplary embodiment, the method includes: (k) contacting the compound formed in step (a) with a Sia-transferase and a Sia donor under conditions appropriate for the Sia-transferase to transfer a Sia moiety from the donor to the compound formed in step (a).

The method also optionally includes: (l) repeating step (k) a selected number of times, thereby forming a poly(sialic acid) substituent on said compound.

The method of the invention can be practiced upon both acylated gangliosides and lyso-gangliosides. The lyso-gangliosides can be acylated at any intermediate point during the reaction cycle leading to the final product, or it can be acylated after the carbohydrate structure is fully in place.

Exemplary compounds formed by the method of the invention set forth above include the gangliosides GM₂, GM₁, GD₁ₐ. GT₁ₐ, Fuc-GM₁, GD₃, GD₂, GD_{1b}, GT_{1b}, GQ_{1b}, GM_{1b}, GD_{1α}, GT_{1β}, GQ_{1B}, GT₃, GT₂, GT_{1c}, GQ_{1c}, globosides (e.g., globo-H, etc.) and polysialylated lactose.

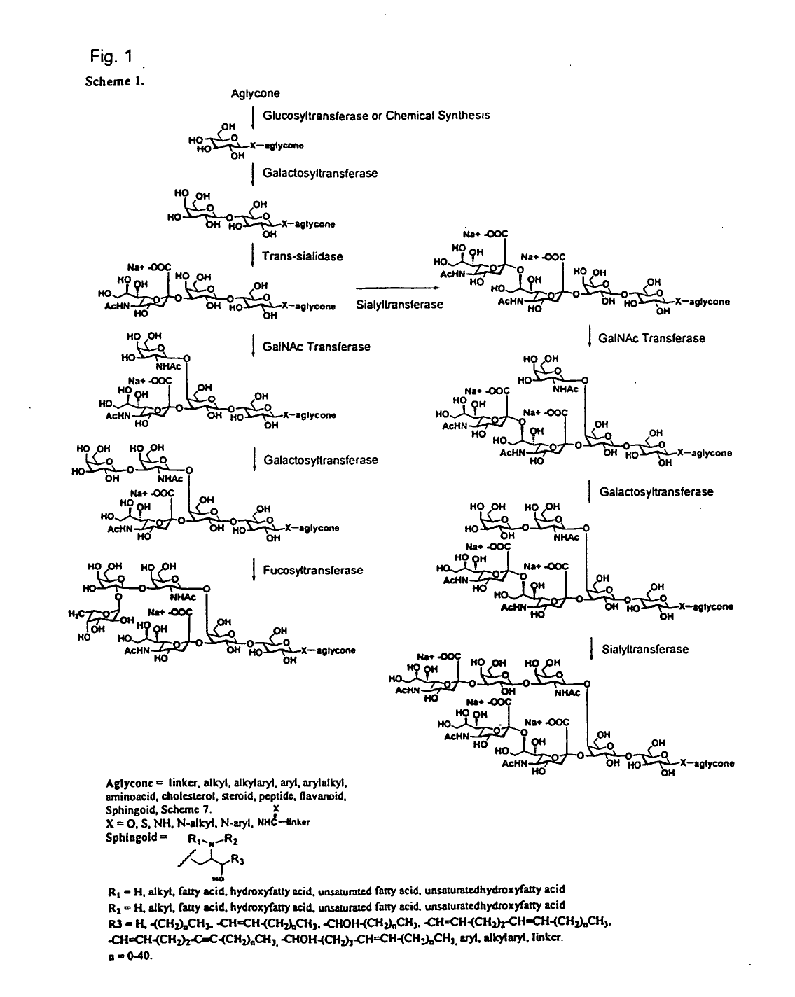

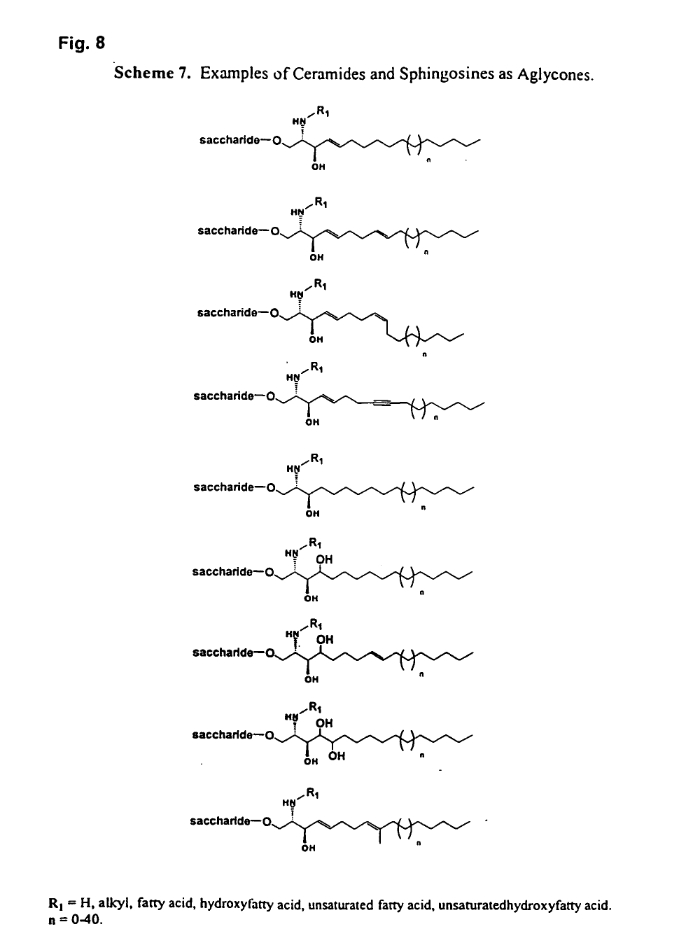

The methods of the invention are further understood by reference to the schemes appended hereto as FIG. 1-FIG. 9. The figures set forth representative syntheses according to the methods of the invention.

With reference to FIG. 1, a substrate (aglycone) is functionalized with glucose either enzymatically (glucosyltransferase) or chemically. The glucosyl derivative is treated with a galactosyltransferase and the galactosylated compound is sialylated using a trans-sialidase. In Pathway 1, GalNAc is appended to galactose residue of the sialylated species. Galactose is conjugated to the GalNAc moiety via a galactosyltransferase, and the Gal residue is fucosylated by the action of a fucosyltransferase.

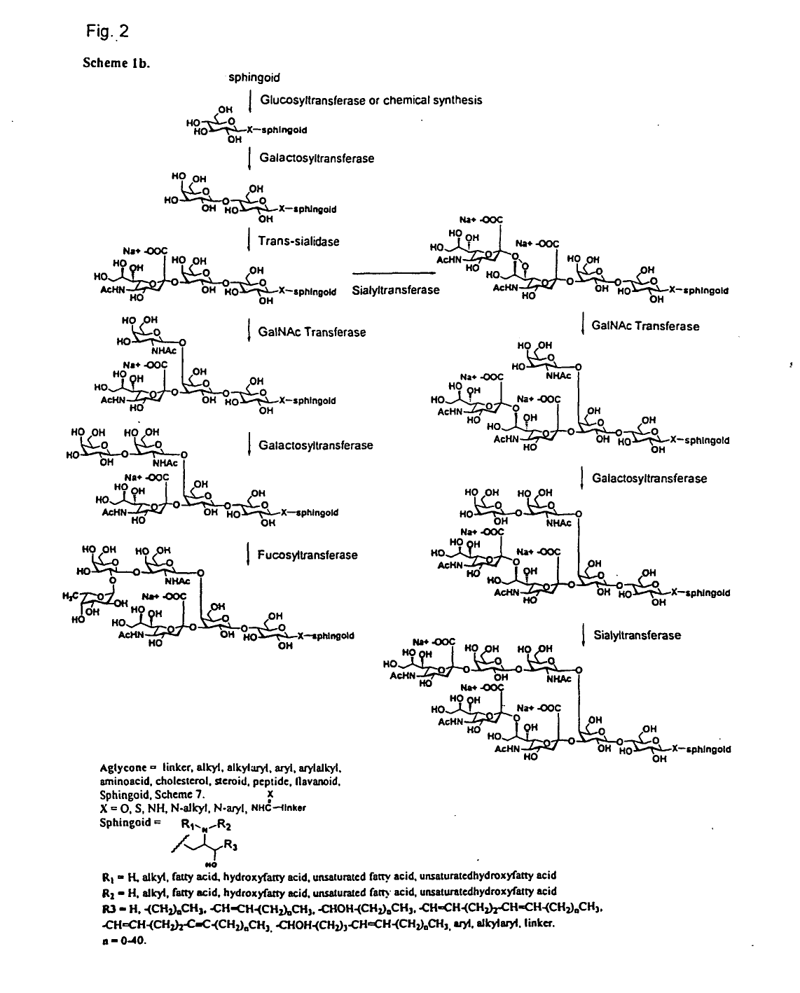

In Pathway 2 of FIG. 2, the sialylated substrate is further sialylated by the addition, using a sialyltransferase, of a sialyl group to the existing sialic acid moiety. The Gal residue is modified with a GalNAc using a GalNAc-transferase. A galactose residue is conjugated to the GalNAc using a galactosyltransferase. The sialic acid moiety is sialylated using a sialyltransferase.

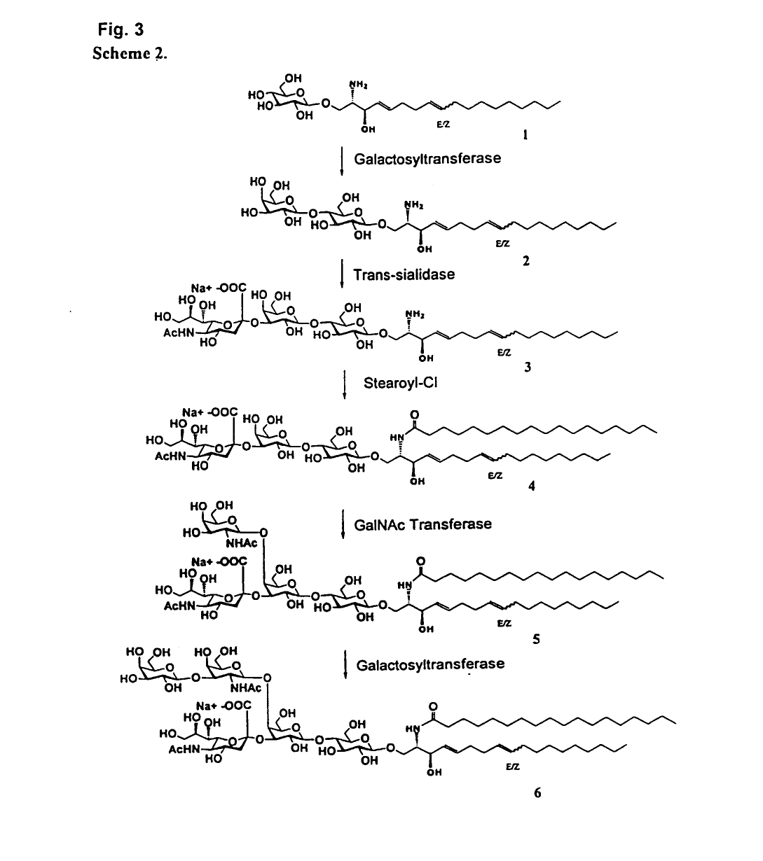

FIG. 3 sets forth an exemplary synthesis of a ganglioside, and sphingosine and ceramide analogues thereof using a method of the invention. Thus, glucosyl sphingoid 1 is galactosylated using a galactosyltransferase. The resulting Glu-Gal sphingoid 2 is sialylated with a trans-sialidase. The primary amine of sialylated sphingoid moiety 3 is acylated with stearoyl chloride, producing the corresponding ceramide 4, which is in turn reacted with GalNAc in the presence of a GalNAc-transferase, forming 5. Compound 5 is contacted with a galactosyltransferase in the presence of a Gal donor to produce compound 6.

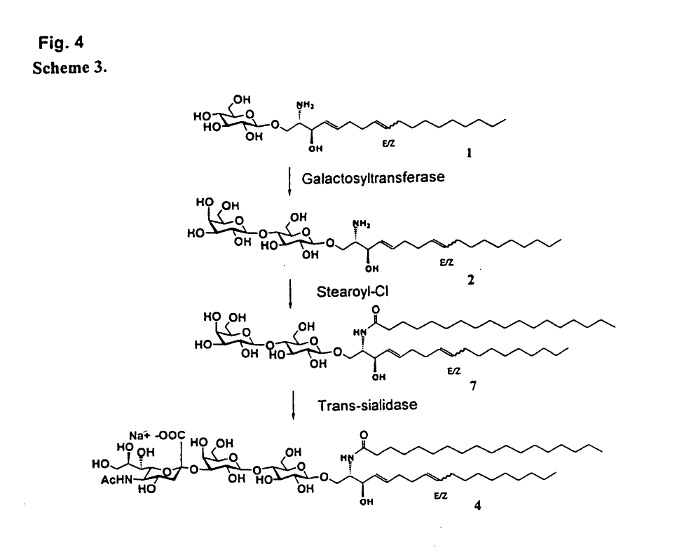

FIG. 4 provides another exemplary synthesis of a ganglioside according to a method of the invention. Thus, the primary amine of the sphingosine moiety of 1 is acylated with stearoyl chloride, producing ceramide 7, which is sialylated by a trans-sialidase, forming 4.

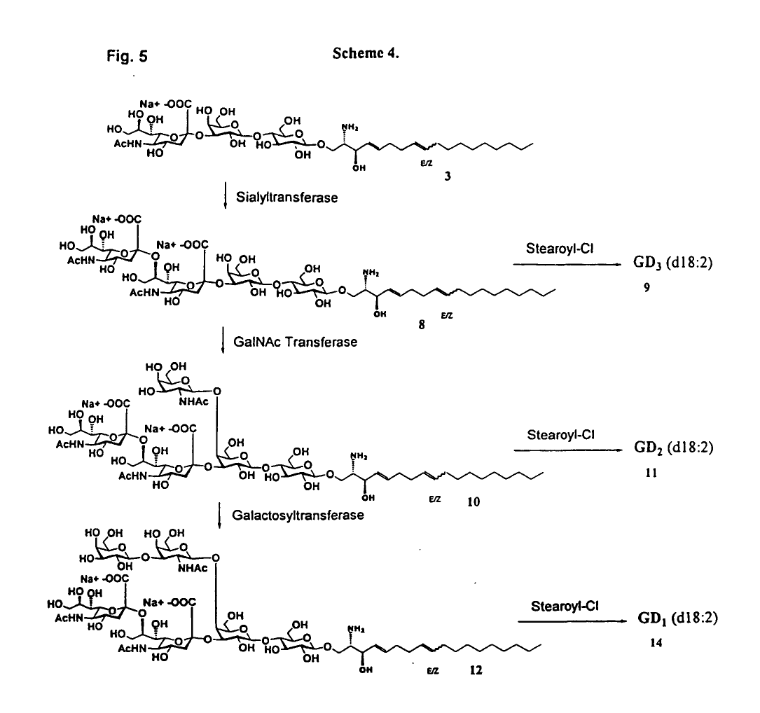

FIG. 5 is a series of schemes to selected gangliosides prepared by methods of the invention. Compound 3 is sialylated with a sialyltransferase, forming compound 8. The amine of compound 8 is acylated with stearoyl chloride to provide GD₃ 9. Alternatively, compound 8 is treated with a GalNAc transferase and a GalNAc donor to produce compound 10, which is acylated with stearoyl chloride to form GD₂ 11. Alternatively, compound 10 is galactosylated, forming 12, which is acylated with stearoyl chloride to produce GD₁ 14.

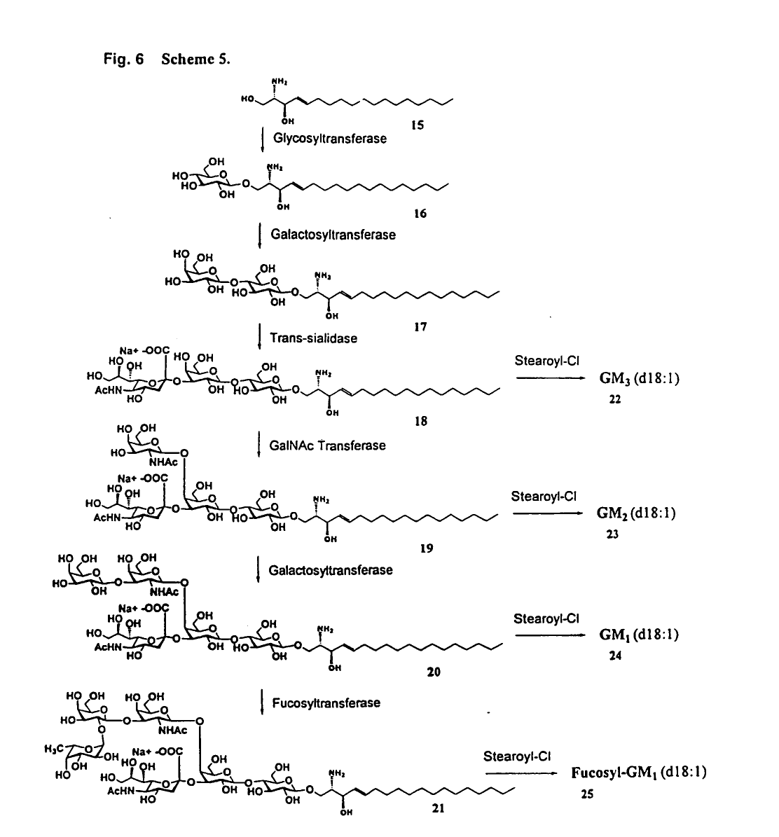

The scheme of FIG. 6 set forth additional exemplary routes to gangliosides using the methods of the invention. Sphingoid 15 is glucosylated, forming 16, to which a galactosyl residue is added, forming 17. Compound 17 is sialylated with a trans-sialidase to form 18, which is optionally acylated at the primary amine with stearoyl chloride to provide GM₃ 22. Alternatively, 17 is treated with a GalNAc transferase and a GalNAc donor to produce 19, which is optionally acylated to provide GM₂ 23. Alternatively, 19 is galactosylated, forming 20, which is optionally acylated to provide GM₁ 24. Alternatively, 20 is fucosylated to form 21, which is optionally acylated, yielding fucosyl-GM₁ 25.

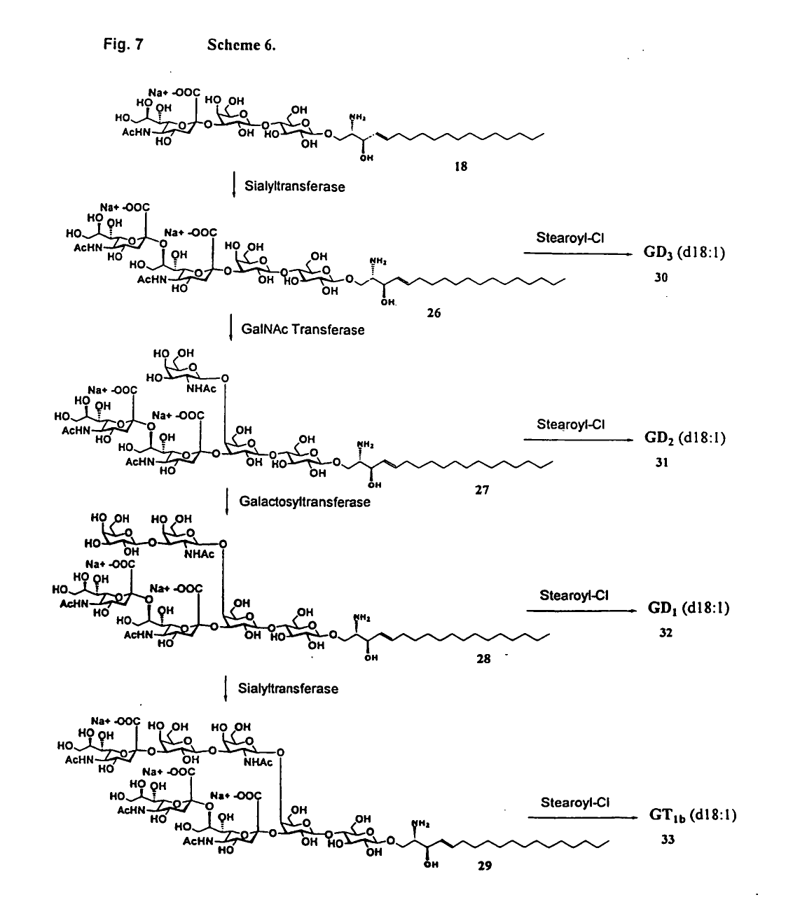

FIG. 7 sets forth exemplary routes using methods of the invention to form gangliosides. Sphingosine 18 is sialylated to 26 using a sialyltransferase. Compound 26 is optionally acylated at the primary amine with stearoyl chloride to form GD3 30. Alternatively, 26 is treated with a GalNAc transferase and a GalNAc donor, forming 27. Compound 27 is galactosylated, forming 28, which is sialylated using a sialyltransferase. Each of compounds 27, 28 and 29 can be acylated with stearoyl chloride to form GD₂ (31), GD₁ (32) or GT_{1b} (33), respectively.

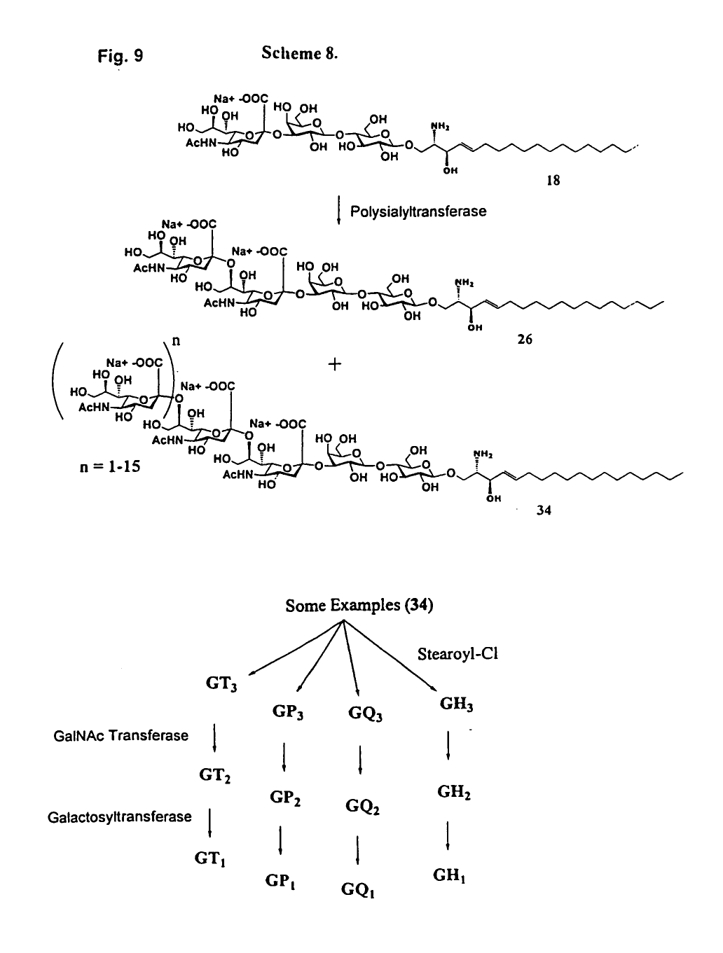

FIG. 9 provides a scheme for preparing polysialylated sphingosines according to a method of the invention. The sphingosines are optionally acylated to form the corresponding ceramide.

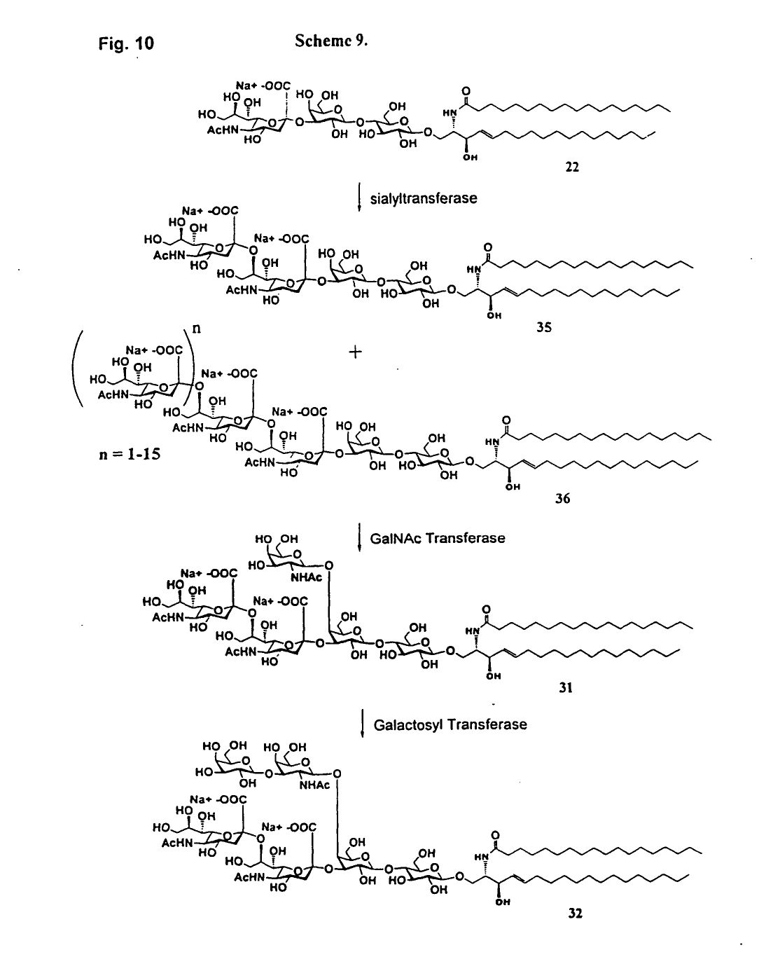

FIG. 10 sets forth an exemplary scheme in which the method of the invention is practiced on an intact ceramide substrate. Ceramide 22 is sialylated providing a mixture of polysialylated species, e.g., 35 and 36, to which GalNAc is conjugated, affording 31. Compound 31 is galactosylated, affording compound 32.

The methods provided by the invention for attaching saccharide residues to substrates can, unlike previously described glycosylation methods provide a population of a substrate in which the members have a substantially uniform glycosylation pattern. Thus, in preferred embodiments, the population of substrates is substantially monodisperse vis-a-vis the glycosylation pattern of each member of the population. After application of the methods of the invention, a desired saccharide residue (e.g., a fucosyl residue) will be attached to a high percentage of acceptor moieties.

The invention also provides a method for reproducing a known glycosylation pattern on a substrate. The method includes glycosylating the substrate to a preselected (i.e., known) level, at which point the glycosylation is stopped. In a particularly preferred embodiment, the substrate is fucosylated to a known level. The method of the invention is of particular use in preparing compositions that are replicas of therapeutic agents, which are presently used clinically or are advanced in clinical trials.

The methods are also practical for large-scale production of modified substrates, including both pilot scale and industrial scale preparations. Thus, the methods of the invention provide a practical means for large-scale preparation of substrates having a selected glycosylation pattern. The processes provide an increased and consistent level of a desired glycoform on substrates present in a composition.

The present invention also provides kits for practicing the methods of the invention. The kits will generally include one or more enzyme of use in practicing the method of the invention and directions for practicing the method of the invention.

The Substrates

The methods of the invention can be practiced using any substrate that includes a suitable acceptor moiety for a glycosyltransferase, a trans-sialidase, and the like. Exemplary substrates include, but are not limited to, sphingosine and its analogues, ceramide and its analogues, peptides, gangliosides and other biological structures (e.g., glycolipids, whole cells, and the like that can be modified by the methods of the invention include any a of a number substrates and carbohydrate structures on cells known to those skilled in the art.

In an exemplary embodiment, the method of the invention utilizes a substrate wherein the structure of X¹ is set forth in Formula V: 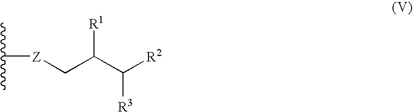
in which Z is selected from O, S and NR⁵. The symbols R¹ and R² independently represent NHR⁴, SR⁴, OR⁴, OCOR⁴, OC(O)NHR⁴, NHC(O)OR⁴, OS(O)₂OR⁴, C(O)R⁴, NHC(O)R⁴, detectable labels, or targeting moieties. R⁴ and R⁵ are members independently selected from H, substituted or unsubstituted alkyl, substituted or unsubstituted heteroalkyl, a detectable labels or a targeting moiety. R³ is selected from substituted or unsubstituted alkyl and substituted or unsubstituted heteroalkyl groups. In an exemplary embodiment, R³ includes at least two degrees of unsaturation. The unsaturation may be present in the form of at least two double bonds or at least one triple bond.

In a still further exemplary embodiment, the structure of X¹ is set forth in Formula VI: 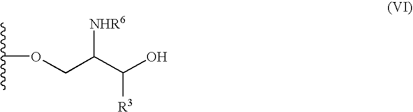
wherein R⁶ is a member selected from H, C(O)R⁷, detectable labels, and targeting moieties; and R⁷ is a member selected from substituted or unsubstituted alkyl, substituted or unsubstituted heteroalkyl, detectable labels and targeting moieties. R³ is generally as described above.

In another exemplary embodiment, the substrate is acylated. The acylation step may occur prior to beginning to assemble the carbohydrate moiety, at any intermediate point during the enzymatic reaction scheme used to assemble the carbohydrate, or after the carbohydrate moiety is fully assembled. For example, when a substrate according to Formula V is utilized and R¹ is a member selected from NH₂, OH and SH, the substrate is optionally acylated at R¹. Methods for acylating lysogangliosides are known in the art, see, for example, “Lysogangliosides: Synthesis and Use in Preparing Labeled Gangliosides” by Gunther Schwarzmann and Konrad Sandhoff in METHODS IN ENZYMOLOGY, Vol. 138, pp. 319-341 (1987).

Acylation according to the described procedure can be carried out in the conventional way, for example, by reacting the starting products with an acylating agent, particularly with a reactive functional derivative of the acid, whose residue is to be introduced. Exemplary reactive functional derivatives of the acid include halides, anhydrides, and active esters. The acylation may be carried out in the presence of a base, (e.g., TEA, pyridine or collidine). Acylation is optionally carried out under anhydrous conditions, at room temperature or with heating. The Schotten-Baumann method may also be used to effect acylation under aqueous conditions in the presence of an inorganic base. In some cases it is also possible to use the esters of the acids as reactive functional derivatives. For acylation, it is possible to also use methods involving activated carboxy derivatives, such as are known in peptide chemistry, for example using mixed anhydrides or derivatives obtainable with carbodiimides or isoxazole salts.

Exemplary methods of acylation include: (1) reaction of the lysoganglioside derivative with the azide of the acid; (2) reaction of the lysoganglioside derivative with an acylimidazole of the acid obtainable from the acid with N,N′-carbonyldiimidazole; (3) reaction of the lysoganglioside derivative with a mixed anhydride of the acid and of trifluoro-acetic acid; (4) reaction of the lysoganglioside derivative with the chloride of the acid; (5) reaction of the lysoganglioside derivative with the acid in the presence of a carbodiimide (such as dicyclohexylcarbodiimide) and optionally of a substance such as 1-hydroxybenzotriazol; (6) reaction of the lysoganglioside derivative with the acid by heating; (7) reaction of the lysoganglioside derivative with a methyl ester of the acid at a high temperature; (8) reaction of the lysoganglioside derivative with a phenol ester of the acid, such as an ester with para-nitrophenol; and (9) reaction of the lysoganglioside derivative with an ester derived from the exchange between a salt of the acid and 1-methyl-2-chloropyridine iodide or similar products.

The acids may be derived from saturated or unsaturated, branched- or straight-chain substituted or unsubstituted alkyl acids, substituted or unsubstituted fatty acids (e.g hydroxy fatty acids). The acyl group may include the substructures: —(CH₂)ₚCH₃, —CH═CH—(CH₂)ₚCH₃, —CHOH—(CH₂)ₚCH₃, —CH═CH—(CH₂)₂—CH═CH—(CH₂)ₚCH₃, —CH═CH—(CH₂)₂—C≡C—(CH₂)ₚCH₃, —CHOH—(CH₂)₃—CH═CH—(CH₂)ₚCH₃, aryl, alkylaryl, or linker, where p is 0-40. In general, the length of the acyl component is preferably from 8 to 25 carbons, more preferably 10-20, and more preferably still from 16 to 18 carbons.

In the particular case of acyl groups derived from acids containing free hydroxy, mercapto, carboxy groups, or primary or secondary amino groups, it is generally preferable to protect such groups during the acylation reaction. Methods for protecting such groups are available in the art. Such protective groups should be easily eliminated at the end of the reaction. Exemplary protecting groups include the phthaloyl group and the benzyloxycarbonyl group, which serves to advantage for the protection of the amino group. Thus, for example, in the preparation of derivatives containing γ-amino butyric acid, a derivative, of this acid is first prepared, where the amino group is bound to the phthaloyl group, and after acylation with the lysoganglioside derivative the phthaloyl group is eliminated by hydrazinolysis. The benzyloxycarbonyl group can be eliminated by hydrogenolysis. This residue may also serve for the protection of the hydroxy groups. The carboxy group can be protected by esterification, for example, with the alcohols used in peptide chemistry.

The Compounds

The invention also provides compounds in which the alkyl portion of the substrate (e.g., R³ in Formulae V or VI) includes two or more degrees of unsaturation. This aspect of the invention is exemplified by sphingosines and ceramides in which the alkyl group has at least two double bonds, or at least one triple bond.

Exemplary compounds of the invention include: 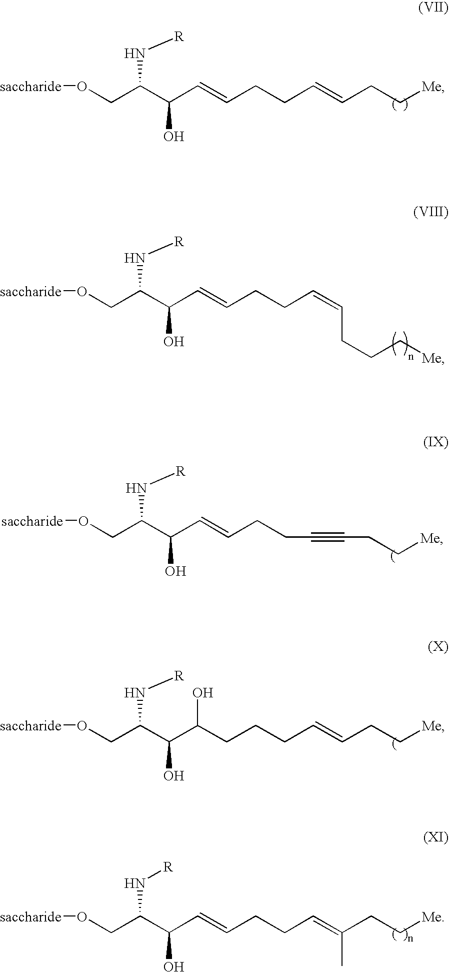
In which R is H, substituted or unsubstituted alkyl, or acyl derived from an acid as discussed above. The symbol n represents an integer from 0-40; preferably, n=6 or 7 (such that for example, the sphingosine base is d18:2 (e.g., trans trans), d18:2 (e.g., trans cis), d18:1:1, t18:1, or d18:2:9 methyl), and R═H or an acyl group derived from a fatty acide, e.g., stearic or palmitic acid.

In other exemplary embodiments, R is an acyl moiety derived from a fatty acid selected from the group consisting of laurate, myristate, palmitate, stearate, arachidate, behenate, lignocerate, palmitoleate, oleate, elaidate, linoleate, linolenate, and arachidonate, or their alpha-hydroxy derivatives. As used herein, the term “fatty acids” refers to those acids that possess a hydrocarbon chain and a terminal carboxyl group, and have the formula CH₃(CH₂)ₙCOOH, where n=1-24. In particularly preferred embodiments, R is an acyl moiety derived from stearic or palmitic acid.

In another exemplary embodiment, the invention provides a method of preparing inner esters of the compounds in which one or more of the hydroxyl groups of the saccharide part are esterified with one or more carboxy groups of an acid. The method also encompasses the formation of “outer” esters of gangliosides, that is, esters of the carboxy functions of sialic acids with various alcohols of the aliphatic, araliphatic, alicyclic or heterocyclic series. Also encompassed are amides of the sialic acids. Methods to prepare each of these derivatives are known in the art. See, for example, U.S. Pat. No. 4,713,374.

The invention also provides methods to prepare metal or organic base salts of the ganglioside compounds according to the present invention having free carboxy functions, and these also form part of the invention. It is possible to prepare metal or organic base salts of other derivatives of the invention too, which have free acid functions, such as esters or peracylated amides with dibasic acids. Also forming part of the invention are acid addition salts of ganglioside derivatives, which contain a basic function, such as a free amino function, for example, esters with aminoalcohols. Of the metal or organic base salts particular mention should be made of those which can be used in therapy, such as salts of alkali or alkaline earth metals, for example, salts of potassium, sodium, ammonium, calcium or magnesium, or of aluminum, and also organic base salts, for example of aliphatic or aromatic or heterocyclic primary, secondary or tertiary amines, such as methylamine, ethylamine, propylamine, piperidine, morpholine, ephedrine, furfurylamine, choline, ethylenediamine and aminoethanol. Of those acids which can give acid addition salts of the ganglioside derivatives according to the invention special mention should be made of hydroacids such as hydrochloric acid, hydrobromic acid, phosphoric acid, sulfuric acid, lower aliphatic acids with a maximum of 7 carbon atoms, such as formic, acetic or propionic acids, succinic and maleic acids. Acids or bases, which are not therapeutically useful, such as picric acid, can be used for the purification of the ganglioside derivatives of the invention and also form part of the invention.

In addition to originating a synthesis of the invention with a substrate that includes neither glycosyl residues or acyl moieties, a synthesis of the invention may originate with a lysoganglioside that is a precursor to the desired ganglioside. Lysogangliosides can be obtained from gangliosides by enzymatic deacylation of the nitrogen with ceramide deacylase (see, J. Biochem. 103: 1 (1988)). The de-N-acyl-lysogangliosides which can also be used as starting products are obtainable from gangliosides with alkaline hydrolyzing agents, for example hydroxides of tetraalkylammonium, potassium hydrate and others (see, Biochemistry 24: 525, (1985); J. Biol. Chem. 255: 7657, (1980); Biol. Chem. Hoppe Seyler 367: 241 (1986); Carbohydr. Res. 179: 393 (1988); Biochem. Biophys. Res. Comm. 147: 127 (1987)).

The Enzymes

a. Glycosyltransferases and Methods for Preparing Substrates having Selected Glycosylation Patterns

The methods of the invention utilize glycosyltransferases (e.g., fucosyltransferases) that are selected for their ability to produce saccharides having a selected glycosylation pattern. For example, glycosyltransferases are selected that not only have the desired specificity, but also are capable of glycosylating a high percentage of desired acceptor groups in the substrate. It is preferable to select the glycosyltransferase based upon results obtained using an assay system that employs an oligosaccharide acceptor moiety, e.g., a soluble oligosaccharide or an oligosaccharide that is attached to a relatively short peptide. In certain embodiments, the glycosyltransferase is a fusion protein. Exemplary fusion proteins include glycosyltransferases that exhibit the activity of two different glycosyltransferases (e.g., sialyltransferase and fucosyltransferase). Other fusion proteins will include two different variations of the same transferase activity (e.g., FucT-VI and FucT-VII). Still other fusion proteins will include a domain that enhances the utility of the transferase activity (e.g, enhanced solubility, stability, turnover, etc.).

A number of methods of using glycosyltransferases to synthesize desired oligosaccharide structures are known and are generally applicable to the instant invention. Exemplary methods are described, for instance, WO 96/32491, Ito et al., Pure Appl. Chem. 65: 753 (1993), and U.S. Pat. Nos. 5,352,670, 5,374,541, and 5,545,553.

Glycosyltransferases catalyze the addition of activated sugars (donor NDP-sugars), in a step-wise fashion, to a substrate (e.g., protein, glycopeptide, lipid, glycolipid or to the non-reducing end of a growing oligosaccharide). A very large number of glycosyltransferases are known in the art.

The method of the invention may utilize any glycosyltransferase, provided that it can add the desired glycosyl residue at a selected site. Examples of such enzymes include Leloir pathway glycosyltransferase, such as galactosyltransferase, N-acetylglucosaminyltransferase, N-acetylgalactosaminyltransferase, fucosyltransferase, sialyltransferase, mannosyltransferase, xylosyltransferase, glucurononyltransferase and the like.

The present invention is practiced using a trans-sialidase and a combination of glycosyltransferases. For example, one can use a combination of a sialyltransferase and a galactosyltransferase in addition to the trans-sialidase. In those embodiments using more than one enzyme, more than one enzyme and the appropriate glycosyl donors are optionally combined in an initial reaction mixture. Alternatively, the enzymes and reagents for a subsequent enzymatic reaction are added to the reaction medium once the previous enzymatic reaction is complete or nearly complete. By conducting two enzymatic reactions in sequence in a single vessel, overall yields are improved over procedures in which an intermediate species is isolated. Moreover, cleanup and disposal of extra solvents and by-products is reduced.

Glycosyltransferases that can be employed in the methods of the invention include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, glucuronic acid transferases, galacturonic acid transferases, and oligosaccharyltransferases. Suitable glycosyltransferases include those obtained from eukaryotes, as well as from prokaryotes.

For enzymatic saccharide syntheses that involve glycosyltransferase reactions, glycosyltransferase can be cloned, or isolated from any source. Many cloned glycosyltransferases are known, as are their polynucleotide sequences. See, e.g., “The WWW Guide To Cloned Glycosyltransferases,” (http://www.vei.co.uk/TGN/gt_guide.htm). Glycosyltransferase amino acid sequences and nucleotide sequences encoding glycosyltransferases from which the amino acid sequences can be deduced are also found in various publicly available databases, including GenBank, Swiss-Prot, EMBL, and others.

DNA encoding the glycosyltransferases may be obtained by chemical synthesis, by screening reverse transcripts of mRNA from appropriate cells or cell line cultures, by screening genomic libraries from appropriate cells, or by combinations of these procedures. Screening of mRNA or genomic DNA may be carried out with oligonucleotide probes generated from the glycosyltransferases gene sequence. Probes may be labeled with a detectable group such as a fluorescent group, a radioactive atom or a chemiluminescent group in accordance with known procedures and used in conventional hybridization assays. In the alternative, glycosyltransferases gene sequences may be obtained by use of the polymerase chain reaction (PCR) procedure, with the PCR oligonucleotide primers being produced from the glycosyltransferases gene sequence. See, U.S. Pat. No. 4,683,195 to Mullis et al. and U.S. Pat. No. 4,683,202 to Mullis.

The glycosyltransferase may be synthesized in host cells transformed with vectors containing DNA encoding the glycosyltransferase. A vector is a replicable DNA construct. Vectors are used either to amplify DNA encoding the glycosyltransferases enzyme and/or to express DNA, which encodes the glycosyltransferases enzyme. An expression vector is a replicable DNA construct in which a DNA sequence encoding the glycosyltransferases enzyme is operably linked to suitable control sequences capable of effecting the expression of the glycosyltransferase in a suitable host. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences that control the termination of transcription and translation. Amplification vectors do not require expression control domains. All that is needed is the ability to replicate in a host, usually conferred by an origin of replication, and a selection gene to facilitate recognition of transform ants.

Examples of suitable glycosyltransferases for use in the preparation of the compositions of the invention are described herein. One can readily identify other suitable glycosyltransferases by reacting various amounts of each enzyme (e.g., 1-100 mU/mg protein) with a substrate (e.g., at 1-10 mg/ml) to which is linked an oligosaccharide that has a potential acceptor site for the glycosyltransferase of interest. The abilities of the glycosyltransferases to add a sugar residue at the desired site are compared. Glycosyltransferases showing the ability to glycosylate the potential acceptor sites of substrate-linked oligosaccharides more efficiently than other glycosyltransferases having the same specificity are suitable for use in the methods of the invention.

For some embodiments, it is advantageous to use a glycosyltransferase that achieves the desired glycoform using a low ratio of enzyme units to substrate. In some embodiments, the desired extent of glycosylation will be obtained using about 50 mU or less of glycosyltransferase per mg of substrate. To obtain a lower cost of enzyme, less than about 40 mU of glycosyltransferase can be used per mg of substrate, even more preferably, the ratio of glycosyltransferase to substrate will be less than or equal to about 35 mU/mg, and more preferably about 25 mU/mg or less. Most preferably from an enzyme cost standpoint, the desired extent of a desired glycosylation will be obtained using less than about 10 mU/mg glycosyltransferase per mg substrate. Typical reaction conditions will have glycosyltransferase present at a range of about 5-25 mU/mg of substrate, or 10-50 mU/ml of reaction mixture with the substrate present at a concentration of at least about 1-2 mg/ml. In a multi-enzyme reaction, these amounts of enzyme can be increased proportionally to the number of glycosyltransferases, sulfotransferases, or trans-sialidases.

In other embodiments, however, it is desirable to use a greater amount of enzyme. For example, to obtain a faster rate of reaction, one can increase the amount of enzyme by about 2-10-fold. The temperature of the reaction can also be increased to obtain a faster reaction rate. A temperature of about 30 to about 37° C., for example, is suitable.

The efficacy of the methods of the invention can be enhanced through use of recombinantly produced glycosyltransferases. Recombinant production enables production of glycosyltransferases in the large amounts that are required for large-scale substrate modification. Deletion of the membrane-anchoring domain of glycosyltransferases, which renders the glycosyltransferases soluble and thus facilitates production and purification of large amounts of glycosyltransferases, can be accomplished by recombinant expression of a modified gene encoding the glycosyltransferases. For a description of methods suitable for recombinant production of glycosyltransferases see, U.S. Pat. No. 5,032,519.

Also provided by the invention are glycosylation methods in which the target substrate is immobilized on a solid support. The term “solid support” also encompasses semi-solid supports. Preferably, the target substrate is reversibly immobilized so that the substrate can be released after the glycosylation reaction is completed. Suitable matrices are known to those of skill in the art. Ion exchange, for example, can be employed to temporarily immobilize a substrate on an appropriate resin while the glycosylation reaction proceeds. A ligand that specifically binds to the substrate of interest can also be used for affinity-based immobilization. Antibodies that bind to a substrate of interest are suitable. Dyes and other molecules that specifically bind to a substrate of interest that is to be glycosylated are also suitable.

In an exemplary embodiment, all of the enzymes used, with the exception of the trans-sialidase, are glycosyltransferases: In another exemplary embodiment, one or more enzymes is a glycosidase.

1. Fucosyltransferase Reactions

Many saccharides require the presence of particular fucosylated structures in order to exhibit biological activity. Intercellular recognition mechanisms often require a fucosylated oligosaccharide. For example, a number of proteins that function as cell adhesion molecules, including P-selectin, E-selectin, bind specific cell surface fucosylated carbohydrate structures, for example, the sialyl Lewis x and the sialyl Lewis a structures. In addition, the specific carbohydrate structures that form the ABO blood group system are fucosylated. The carbohydrate structures in each of the three groups share a Fucα1,2Galβ1-dissacharide unit. In blood group O structures, this disaccharide is the terminal structure. The group A structure is formed by an α1,3 GalNAc transferase that adds a terminal GalNAc residue to the dissacharide. The group B structure is formed by an α1,3 galactosyltransferase that adds terminal galactose residue. The Lewis blood group structures are also fucosylated. For example the Lewis x and Lewis a structures are Galβ1,4(Fucα1,3)GlcNac and Galβ1,4(Fucα1,4)GlcNac, respectively. Both these structures can be further sialylated (NeuAcα2,3-) to form the corresponding sialylated structures. Other Lewis blood group structures of interest are the Lewis y and b structures which are Fucα1,2Galβ1,4(Fucα1,3)GlcNAcβ-OR and Fucα1,2Galβ1,3(Fucα1,4)GlcNAc-OR, respectively. For a description of the structures of the ABO and Lewis blood group stuctures and the enzymes involved in their synthesis see, Essentials of Glycobiology, Varki et al. eds., Chapter 16 (Cold Spring Harbor Press, Cold Spring Harbor, N.Y., 1999).

Fucosyltransferases have been used in synthetic pathways to transfer a fucose unit from guanosine-5′-diphosphofucose to a specific hydroxyl of a saccharide acceptor. For example, Ichikawa prepared sialyl Lewis-X by a method that involves the fucosylation of sialylated lactosamine with a cloned fucosyltransferase (Ichikawa et al., J. Am. Chem. Soc. 114: 9283-9298 (1992)). Lowe has described a method for expressing non-native fucosylation activity in cells, thereby producing fucosylated glycoproteins, cell surfaces, etc. (U.S. Pat. No. 5,955,347).

In one embodiment, the methods of the invention are practiced by contacting a substrate, having an acceptor moiety for a fucosyltransferase, with a reaction mixture that includes a fucose donor moiety, a fucosyltransferase, and other reagents required for fucosyltransferase activity. The substrate is incubated in the reaction mixture for a sufficient time and under appropriate conditions to transfer fucose from the fucose donor moiety to the fucosyltransferase acceptor moiety. In preferred embodiments, the fucosyltransferase catalyzes the fucosylation of at least 60% of the fucosyltransferase respective acceptor moieties in the composition.

A number of fucosyltransferases are known to those of skill in the art. Briefly, fucosyltransferases include any of those enzymes, which transfer L-fucose from GDP-fucose to a hydroxy position of an acceptor sugar. In some embodiments, for example, the acceptor sugar is a GlcNAc in a Galβ(1→3,4)GlcNAc group in an oligosaccharide glycoside. Suitable fucosyltransferases for this reaction include the known Galβ(1→3,4)GlcNAcα(1→3,4)fucosyltransferase (FucT-III E.C. No. 2.4.1.65) which is obtained from human milk (see, e.g., Palcic et al., Carbohydrate Res. 190:1-11 (1989); Prieels, et al., J. Biol. Chem. 256:10456-10463 (1981); and Nunez, et al., Can. J. Chem. 59:2086-2095 (1981)) and the βGal(1→4)βGlcNAc α(1→3)fucosyltransferases (FucT-IV, FucT-V, FucT-VI, and FucT-VII, E.C. No. 2.4.1.65) which are found in human serum. A recombinant form of βGal(1→3,4)βGlcNAc α(1→3,4)fucosyltransferase is also available (see, Dumas, et al., Bioorg. Med. Letters 1: 425-428 (1991) and Kukowska-Latallo, et al., Genes and Development 4: 1288-1303 (1990)). Other exemplary fucosyltransferases include α1,2 fucosyltransferase (E.C. No. 2.4.1.69). Enzymatic fucosylation may be carried out by the methods described in Mollicone et al., Eur. J. Biochem. 191:169-176 (1990) or U.S. Pat. No.5,374,655; an α1,3-fucosyltransferase from Schistosoma mansoni (Trottein et al. (2000) Mol. Biochem. Parasitol. 107:,279-287); and an (α1,3 fucosyltransferase IX (nucleotide sequences of human and mouse FucT-IX are described in Kaneko et al. (1999) FEBS Lett. 452: 237-242, and the chromosomal location of the human gene is described in Kaneko et al. (1999) Cytogenet. Cell Genet. 86: 329-330. Recently reported α1,3-fucosyltransferases that use an N-linked GlcNAc as an acceptor from the snail Lymnaea stagnalis and from mung bean are described in van Tetering et al. (1999) FEBS Lett. 461: 311-314 and Leiter et al. (1999) J. Biol. Chem. 274: 21830-21839, respectively. In addition, bacterial fucosyltransferases such as the α(1,3/4) fucosyltransferase of Helicobacter pylori as described in Rasko et al. (2000) J. Biol. Chem. 275:4988-94, as well as the α1,2-fucosyltransferase of H. Pylori (Wang et al. (1999) Microbiology. 145: 3245-53. See, also Staudacher, E. (1996) Trends in Glycoscience and Glycotechnology, 8: 391-408 for description of fucosyltransferases useful in the invention.

In some embodiments, the fucosyltransferase that is employed in the methods of the invention has an activity of at least about 1 U/mL, usually at least about 5 U/mL.

In other embodiments, fucosyltransferases for use in the methods of the invention include FucT-VII and FucT-VI.

Certain FucT molecules are surprisingly effective at fucosylating substrates. For example, FucT-VI is approximately 8-fold more effective at fucosylating substrates than is FucT-V. Thus, in a preferred embodiment, the invention provides a method of fucosylating an acceptor on a substrate using a fucosyltransferase that provides a degree of fucosylation that is at least about 2-fold greater, more preferably at least about 4-fold greater, still more preferably at least about 6-fold greater, and even more preferably at least about 8-fold greater than is achieved under identical conditions using FucT-V. Presently preferred fucosyltransferases include FucT-VI and FucT-VII.

Specificity for a selected substrate is only the first criterion a preferred fucosyltransferase should satisfy. The fucosyltransferase used in the method of the invention is preferably also able to efficiently fucosylate a variety of substrates, and support scale-up of the reaction to allow the fucosylation of at least about 500 mg of the substrate. More preferably, the fucosyltransferase will support the scale of the fucosylation reaction to allow the synthesis of at least about 1 kg, and more preferably, at least 10 kg of substrate with relatively low cost and infrastructure requirements.

Suitable acceptor moieties for fucosyltransferase-catalyzed attachment of a fucose residue include, but are not limited to, GlcNAc-OR, Galβ1,3GlcNAc-OR, NeuAcα2,3Galβ1,3GlcNAc-OR, Galβ1,4GlcNAc-OR and NeuAcα2,3Galβ1,4GlcNAc-OR, where R is an amino acid, a saccharide, an oligosaccharide or an aglycon group having at least one carbon atom. R is linked to or is part of a substrate. The appropriate fucosyltransferase for a particular reaction is chosen based on the type of fucose linkage that is desired (e.g., α2, α3, or α4), the particular acceptor of interest, and the ability of the fucosyltransferase to achieve the desired high yield of fucosylation. Suitable fucosyltransferases and their properties are described above.

If a sufficient proportion of the substrate-linked oligosaccharides in a composition does not include a fucosyltransferase acceptor moiety, one can synthesize a suitable acceptor. For example, one preferred method for synthesizing an acceptor for a fucosyltransferase involves use of a GlcNAc transferase to attach a GlcNAc residue to a GlcNAc transferase acceptor moiety, which is present on the substrate-linked oligosaccharides. In preferred embodiments a transferase is chosen, having the ability to glycosylate a large fraction of the potential acceptor moieties of interest. The resulting GlcNAcβ-OR can then be used as an acceptor for a fucosyltransferase.

The resulting GlcNAcβ-OR moiety can be galactosylated prior to the fucosyltransferase reaction, yielding, for example, a Galβ1,3GlcNAc-OR or Gal β1,4GlcNAc-OR residue. In some embodiments, the galactylation and fucosylation steps can be carried out simultaneously. By choosing a fucosyltransferase that requires the galactosylated acceptor, only the desired product is formed. Thus, this method involves:
(a) galactosylating a compound of the formula GlcNAcβ-OR with a galactosyltransferase in the presence of a UDP-galactose under conditions sufficient to form the compounds Galβ1,4GlcNAcβ-OR or Galβ1,3GlcNAc-OR; and
(b) fucosylating the compound formed in (a) using a fucosyltransferase in the presence of GDP-fucose under conditions sufficient to form a compound selected from:
   Fucα1,2Galβ1,4GlcNAc1β-O1R;
   Fucα1,2Galβ1,3GlcNAc-OR;
   Fucα1,2Galβ1,4GalNAc1β-O1R;
   Fucα1,2Galβ1,3GalNAc-OR;
   Galβ1,4(Fuc1,α3)GlcNAcβ-OR; or
   Galβ1,3(Fucα1,4)GlcNAc-OR.

One can add additional fucose residues to the above structures by including an additional fucosyltransferase, which has the desired activity. For example, the methods can form oligosaccharide determinants such as Fucα1,2Galβ1,4(Fucα1,3)GlcNAcβ-OR and Fucα1,2Galβ1,3(Fucα1,4)GlcNAc-OR. Thus, in another preferred embodiment, the method includes the use of at least two fucosyltransferases. The multiple fucosyltransferases are used either simultaneously or sequentially. When the fucosyltransferases are used sequentially, it is generally preferred that the glycoprotein is not purified between the multiple fucosylation steps. When the multiple fucosyltransferases are used simultaneously, the enzymatic activity can be derived from two separate enzymes or, alternatively, from a single enzyme having more than one fucosyltransferase activity.

2. Sialyltransferases

Oligosaccharide determinants that confer a desired biological activity upon a substrate often are sialylated. Accordingly, the invention provides methods in which a substrate-linked oligosaccharide is sialylated in high yields. In a preferred embodiment, the method produces a population of substrates in which the members have a substantially uniform sialylation pattern. Typically, the saccharide chains on a substrate having sialylated species produced by the methods of the invention will have a greater percentage of terminal galactose residues sialylated than the unaltered substrate. Preferably, greater than about 60%, more preferably greater than about 80% of terminal galactose residues present on the substrate-linked oligosaccharides will be sialylated following use of the methods. More preferably, the methods of the invention will result in greater than about 90% sialylation, and even more preferably greater than about 95% sialylation of terminal galactose residues. Most preferably, essentially 100% of the terminal galactose residues present on the substrates in the composition are sialylated following modification using the methods of the present invention. The methods are typically capable of achieving the desired level of sialylation in about 48 hours or less, and more preferably in about 24 hours or less.

Examples of recombinant sialyltransferases, including those having deleted anchor domains, as well as methods of producing recombinant sialyltransferases, are found in, for example, U.S. Pat. No. 5,541,083. At least 15 different mammalian sialyltransferases have been documented, and the cDNAs of thirteen of these have been cloned to date (for the systematic nomenclature that is used herein, see, Tsuji et al. (1996) Glycobiology 6: v-xiv). These cDNAs can be used for recombinant production of sialyltransferases, which can then be used in the methods of the invention.

The sialylation can be accomplished using either a trans-sialidase or a sialyltransferase, except where a particular determinant requires an α2,6-linked sialic acid, in which case a sialyltransferase is used. The present methods involve sialylating an acceptor for a sialyltransferase or a trans-sialidase by contacting the acceptor with the appropriate enzyme in the presence of an appropriate donor moiety. For sialyltransferases, CMP-sialic acid is a preferred donor moiety. Trans-sialidases, however, preferably use a donor moiety that includes a leaving group to which the trans-sialidase cannot add sialic acid.

Acceptor moieties of interest include, for example, Galβ-OR. In some embodiments, the acceptor moieties are contacted with a sialyltransferase in the presence of CMP-sialic acid under conditions in which sialic acid is transferred to the non-reducing end of the acceptor moiety to form the compound NeuAcα2,3Galβ-OR or NeuAcα2,6Galβ-OR. In this formula, R is an amino acid, a saccharide, an oligosaccharide or an aglycon group having at least one carbon atom. In an exemplary embodiment, Galβ-OR is Galβ1,4GlcNAc-R, wherein R is linked to or is part of a substrate.

In an exemplary embodiment, the method provides a compound that is both sialylated and fucosylated. The sialyltransferase and fucosyltransferase reactions are generally conducted sequentially, since most sialyltransferases are not active on a fucosylated acceptor. FucT-VII, however, acts only on a sialylated acceptor. Therefore, FucT-VII can be used in a simultaneous reaction with a sialyltransferase.

If the trans-sialidase is used to accomplish the sialylation, the fucosylation and sialylation reactions can be conducted either simultaneously or sequentially, in either order. The substrate to be modified is incubated with a reaction mixture that contains a suitable amount of a trans-sialidase, a suitable sialic acid donor substrate, a fucosyltransferase (capable of making an α1,3 or α1,4 linkage), and a suitable fucosyl donor substrate (e.g., GDP-fucose).

Examples of sialyltransferases that are suitable for use in the present invention include ST3Gal III (e.g., a rat or human ST3Gal III), ST3Gal IV, ST3Gal I, ST6Gal I, ST3Gal V, ST6Gal II, ST6GalNAc I, ST6GalNAc II, and ST6GalNAc III (the sialyltransferase nomenclature used herein is as described in Tsuji et al., Glycobiology 6: v-xiv (1996)). An exemplary α(2,3)sialyltransferase referred to as α(2,3)sialyltransferase (EC 2.4.99.6) transfers sialic acid to the non-reducing terminal Gal of a Galβ1→3Glc disaccharide or glycoside. See, Van den Eijnden et al., J. Biol. Chem. 256: 3159 (1981), Weinstein et al., J. Biol. Chem. 257: 13845 (1982) and Wen et al., J. Biol. Chem. 267: 21011 (1992). Another exemplary α2,3-sialyltransferase (EC 2.4.99.4) transfers sialic acid to the non-reducing terminal Gal of the disaccharide or glycoside. see, Rearick et al., J. Biol. Chem. 254: 4444 (1979) and Gillespie et al., J. Biol. Chem. 267: 21004 (1992). Further exemplary enzymes include Gal-β-1,4-GlcNAc α-2,6 sialyltransferase (See, Kurosawa et al. Eur. J. Biochem. 219: 375-381 (1994)). An α2,8-sialyltransferase can also be used to attach a second or multiple sialic acid residues to substrates useful in methods of the invention. A still further example is the alpha2,3-sialyltransferases from Streptococcus agalactiae (ST known as cpsK gene), Haemophilus ducreyi (known as 1st gene), Haemophilus influenza (known as HI0871 gene). See, Chaffin et al., Mol. Microbiol., 45: 109-122 (2002).

TABLE 1
Sialyltransferases which use the Galβ1, 4GlcNAc
sequence as an acceptor substrate

| Sialyltransferase | Source | Sequence(s) formed | Ref. |
|---|---|---|---|
| ST6Gal I | Mammalian | NeuAcα2,6Galβ1,4GlCNAc- | 1 |
| ST3Gal III | Mammalian | NeuAcα2,3Galβ1,4GlCNAc- | 1 |
| | | NeuAcI2,3Galβ1,3GlCNAc- | |
| ST3Gal IV | Mammalian | NeuAcα2,3Galβ1,4GlCNAc- | 1 |
| | | NeuAcα2,3Galβ1,3GlCNAc- | |
| ST6Gal II | Mammalian | NeuAcα2,6Galβ1,4GlCNA | ** |
| ST6Gal II | photobacterium | NeuAcα2,6Galβ1,4GlCNAc- | 2 |
| ST3Gal V | N. meningitides | NeuAcα2,3Galβ1,4GlCNAc- | 3 |
| | N. | | |
| | gonorrhoeae | | |
| ST3Gal I | Mammalian | Neu5Acα2,3Galβ1,3GalNAc | |
| ST3Gal II | Mammalian | Neu5Acα2,3Galβ1,4GlcNAc | |
| ST3Gal IV | Mammalian | Neu5Acα2,3Galβ1,4GlcNAc | |
| | | Neu5Acα2,3Galβ1,3GlcNAc | |
| ST6GalNAc I | Mammalian | Neu5Ac2,6GalNAc | |
| | | Galβ1,3GalNAc(Neu5Acα2, | |
| | | 6) | |
| | | Galβ1,3GalNAc(Neu5Acα2, | |
| | | 6) | |
| | | Neu5Acα2,3Galβ1,3GalNAc- | |
| | | (Neu5Acα2,6) | |
| ST6GalNAc II | Mammalian | Neu5Ac2,6GalNAc | |
| | | Galβ1,3GalNAc(Neu5Acα2, | |
| | | 6) | |

1) Goochee et al., Bio/Technology 9: 1347-1355 (1991)
2) Yamamoto et al., J. Biochem. 120: 104-110 (1996)
3) Gilbert et al., J. Biol. Chem. 271: 28271-28276 (1996)

An example of a sialyltransferase that is useful in the claimed methods is ST3Gal III, which is also referred to as α(2,3)sialyltransferase (EC 2.4.99.6). This enzyme catalyzes the transfer of sialic acid to the Gal of a Galβ1,3GlcNAc or Galβ1,4GlcNAc glycoside (see, e.g., Wen et al., J. Biol. Chem. 267: 21011 (1992); Van den Eijnden et al., J. Biol. Chem. 256: 3159 (1991)) and is responsible for sialylation of asparagine-linked oligosaccharides in glycopeptides. The sialic acid is linked to a Gal with the formation of an α-linkage between the two saccharides. Bonding (linkage) between the saccharides is between the 2-position of NeuAc and the 3-position of Gal. This particular enzyme can be isolated from rat liver (Weinstein et al., J. Biol. Chem. 257: 13845 (1982)); the human cDNA (Sasaki et al. (1993) J. Biol. Chem. 268: 22782-22787; Kitagawa & Paulson (1994) J. Biol. Chem. 269: 1394-1401) and genomic (Kitagawa et al. (1996) J. Biol. Chem. 271: 931-938) DNA sequences are known, facilitating production of this enzyme by recombinant expression. In a preferred embodiment, the claimed sialylation methods use a rat ST3Gal III.

Other exemplary sialyltransferases of use in the present invention include those isolated from Campylobacter jejuni, including the α(2,3) sialyltransferase. See, e.g, WO99/49051. In another embodiment, the invention provides bifunctional sialyltransferase polypeptides that have both an a2,3 sialyltransferase activity and an a2,8 sialyltransferase activity. The bifunctional sialyltransferases, when placed in a reaction mixture with a suitable saccharide acceptor (e.g., a saccharide having a terminal galactose), and a sialic acid donor (e.g., CMP-sialic acid) can catalyze the transfer of a first sialic acid from the donor to the acceptor in an α2,3 linkage. The sialyltransferase then catalyzes the transfer of a second sialic acid from a sialic acid donor to the first sialic acid residue in an α2,8 linkage. This type of Siaα2,8-Siaα2,3-Gal structure is often found in gangliosides. See, for example, EP Pat. App. No. 1147200.

In some embodiments, the sialylation methods used in the invention have increased commercial practicality through the use of bacterial sialyltransferases, either recombinantly produced or produced in the native bacterial cells. Two bacterial sialyltransferases have been recently reported; an ST6Gal II from Photobacterium damsela (Yamamoto et al. (1996) J. Biochem. 120: 104-110) and an ST3Gal V from Neisseria meningitidis (Gilbert et al. (1996) J. Biol. Chem. 271: 28271-28276). The two recently described bacterial enzymes transfer sialic acid to the Galβ1,4GlcNAc sequence on oligosaccharide substrates.

A recently reported viral α2,3-sialyltransferase is also suitable use in the sialylation methods of the invention (Sujino et al. (2000) Glycobiology 10: 313-320). This enzyme, v-ST3Gal I, was obtained from Myxoma virus-infected cells and is apparently related to the mammalian ST3Gal IV as indicated by comparison of the respective amino acid sequences. v-ST3Gal I catalyzes the sialylation of Type I (Galβ1,3-GlcNAcβ1-R), Type II (Galβ1,4GlcNAc-β1-R) and III (Gal β1,3GalNAcβ1-R) acceptors. The enzyme can also transfer sialic acid to fucosylated acceptor moieties (e.g., Lewis^{x} and Lewis^{a}).

3. Galactosyltransferases

In another group of embodiments, the glycosyltransferase is a galactosyltransferase. Exemplary galactosyltransferases include α(1,3) galactosyltransferases (E.C. No. 2.4.1.151, see, e.g., Dabkowski et al., Transplant Proc. 25:2921 (1993) and Yamamoto et al. Nature 345: 229-233 (1990), bovine (GenBank j04989, Joziasse et al., J. Biol. Chem. 264: 14290-14297 (1989)), murine (GenBank m26925; Larsen et al., Proc. Nat'l. Acad. Sci. USA 86: 8227-8231 (1989)), porcine (GenBank L36152; Strahan et al., Immunogenetics 41: 101-105 (1995)). Another suitable α1,3 galactosyltransferase is that which is involved in synthesis of the blood group B antigen (EC 2.4.1.37, Yamamoto et al., J. Biol. Chem. 265: 1146-1151 (1990) (human)).

Also suitable for use in the methods of the invention are β(1,4) galactosyltransferases, which include, for example, EC 2.4.1.90 (LacNAc synthetase) and EC 2.4.1.22 (lactose synthetase) (bovine (D'Agostaro et al., Eur. J. Biochem. 183: 211-217 (1989)), human (Masri et al., Biochem. Biophys. Res. Commun. 157: 657-663 (1988)), murine (Nakazawa et al., J. Biochem. 104: 165-168 (1988)), as well as E.C. 2.4.1.38 and the ceramide galactosyltransferase (EC 2.4.1.45, Stahl et al., J. Neurosci. Res. 38: 234-242 (1994)). Other suitable galactosyltransferases include, for example, α1,2 galactosyltransferases (from e.g., Schizosaccharomyces pombe, Chapell et al., Mol. Biol. Cell 5: 519-528 (1994)). Other 1,4-galactosyltransferases are those used to produce globosides. Both mammalian and bacterial enzymes are of use.

The production of proteins such as the enzyme GalNAc T_{1-XIV} from cloned genes by genetic engineering is well known. See, eg., U.S. Pat. No.4,761,371. One method involves collection of sufficient samples, then the amino acid sequence of the enzyme is determined by N-terminal sequencing. This information is then used to isolate a cDNA clone encoding a full-length (membrane bound) transferase which upon expression in the insect cell line Sf9 resulted in the synthesis of a fully active enzyme. The acceptor specificity of the enzyme is then determined using a semiquantitative analysis of the amino acids surrounding known glycosylation sites in 16 different proteins followed by in vitro glycosylation studies of synthetic peptides. This work has demonstrated that certain amino acid residues are overrepresented in glycosylated peptide segments and that residues in specific positions surrounding glycosylated serine and threonine residues may have a more marked influence on acceptor efficiency than other amino acid moieties.

Other exemplary galactosyltransferases of use in the invention include β1,3-galactosyltransferases. When placed in a suitable reaction medium, the β1,3-galactosyltransferases, catalyze the transfer of a galactose residue from a donor (e.g., UDP-Gal) to a suitable saccharide acceptor (e.g., saccharides having a terminal GalNAc residue). An example of β1,3-galactosyltransferase of the invention is that produced by Campylobacter species, such as C. jejuni. A presently preferred β1,3-galactosyl-transferase of the invention is that of C. jejuni strain OH4384

Exemplary linkages in compounds formed by the method of the invention using galactosyltransferases include: (1) Galβ1→4Glc; (2) Galβ1→4GlcNAc; (3) Galβ1→3GlcNAc; (4) Galβ1→6GlcNAc; (5) Galβ1→3GalNAc; (6) Galβ1→6GalNAc; (7) Galα1→3GalNAc; (8) Galα1→3Gal; (9) Galα1→4Gal; (10) Galβ1→3Gal; (11) Galβ1→4Gal; (12) Galβ1→6Gal; (13) Galβ1→4xylose; (14) Galβ1→1′-sphingosine; (15) Galβ1→1′-ceramide; (16) Galβ1→3 diglyceride; (17) Galβ1→O-hydroxylysine; and (18) Gal-S-cysteine. See, for example, U.S. Pat. Nos. 6,268,193; and 5,691,180.

4. Trans-sialidase

As discussed above, the process of the invention involves at least one step in which a sialic acid moiety is added to a substrate using a trans-sialidase. As used herein, the term “trans-sialidase” refers to an enzyme that catalyzes the addition of a sialic acid to galactose through an α-2,3 glycosidic linkage. Trans-sialidases are found in many Trypanosomy species and some other parasites. Trans-sialidases of these parasite organisms retain the hydrolytic activity of usual sialidase, but with much less efficiency, and catalyze a reversible transfer of terminal sialic acids from host sialoglycoconjugates to parasite surface glycoproteins in the absence of CMP-sialic acid. Trypanosome cruzi, which causes Chagas disease, has a surface trans-sialidase the catalyzes preferentially the transference of α2,3-linked sialic acid to acceptors containing terminal β-galactosyl residues, instead of the typical hydrolysis reaction of most sialidases (Ribeirão et al., Glycobiol. 7: 1237-1246 (1997); Takahashi et al., Anal. Biochem. 230: 333-342 (1995); Scudder et al., J. Biol. Chem. 268: 9886-9891 (1993); and Vandekerckhove et al., Glycobiol. 2: 541-548 (1992)). T. cruzi trans-sialidase (TcTs) has activity towards a wide range of saccharide, glycolipid, and glycoprotein acceptors which terminate with a β-linked galactose residue, and synthesizes exclusively an α2-3 sialosidic linkage (Scudder et al., supra). At a low rate, it also transfers sialic acid from synthetic α-sialosides, such as p-nitrophenyl-α-N-acetylneuraminic acid, but NeuAc2-3Galβ1-4(Fucα1-3)Glc is not a donor-substrate. Modified 2-[4-methylumbelliferone]-α-ketoside of N-acetyl-D-neuraminic acid (4MU-NANA) and several derivatives thereof can also serve as donors for TcTs (Lee & Lee, Anal. Biochem. 216: 358-364 (1994)). Enzymatic synthesis of 3′-sialyl-lacto-N-biose I has been catalyzed by TcTs from lacto-N-biose I as acceptor and 2′-(4-methylumbellyferyl)-α-D-N-acelyneuraminic as donor of the N-acetylneuraminil moiety (Vetere et al., Eur. J. Biochem. 267: 942-949 (2000)). Further information regarding the use of trans-sialidase to synthesize α2,3-sialylated conjugates can be found in European Patent Application No. 0 557 580 A2 and U.S. Pat. No. 5,409,817, each of which is incorporated herein by reference. The intramnolecular trans-sialidase from the leech Macrobdella decora exhibits strict specificity toward the cleavage of terminal Neu5Ac (N-acetylneuraminic acid) α2→3Gal linkage in sialoglycoconjugates and catalyzes an intramolecular trans-sialosyl reaction (Luo et al., J. Mol. Biol. 285: 323-332 (1999). Trans-sialidases primarily add sialic acid onto galactose acceptors, although, they will transfer sialic acid onto some other sugars. Transfer of sialic acid onto GalNAc, however, requires a sialyltransferase. Further information on the use of trans-sialidases can be found in PCT Application No. WO 93/18787; and Vetere et al., Eur. J. Biochem. 247:1083-1090 (1997).

5. GalNAc Transferases

The invention also may utilize β1,4-GalNAc transferase polypeptides. The β1,4-GalNAc transferases, when placed in a reaction mixture, catalyze the transfer of a GalNAc residue from a donor (e.g., UDP-GalNAc) to a suitable acceptor saccharide (typically a saccharide that has a terminal galactose residue). The resulting structure, GalNAc⊕1,4-Gal-, is often found in gangliosides and other sphingoids, among many other saccharide compounds.

An example of a β1,4-GalNAc transferase useful in the present invention is that produced by Campylobacter species, such as C. jejuni. A presently preferred β1,4-GalNAc transferase polypeptide is that of C. jejuni strain OH4384.

Exemplary GalNAc transferases of use in the present invention form the following linkages: (1) (GalNAcα1→3)[(Fucα1→2)]Galβ-; (2) GalNAcα1→Ser/Thr; (3) GalNAcβ1→4Gal; (4) GalNAcβ1→3Gal; (5) GalNAcα1→3GalNAc; (6) (GalNAcβ1→4GlcUAβ1→3)ₙ; (7) (GalNAcβ1→41dUAα1→3-)ₙ; (8) -Manβ→GalNAcαGlcNAcαAsn. See, for example, U.S. Pat. Nos. 6,268,193; and 5,691,180.

6. GlcNAc Transferases

The present invention optionally makes use of GlcNAc transferases. Exemplary N-Acetylglucosaminyltransferases useful in practicing the present invention are able to form the following linkages: (1) GlcNAcβ1→4GlcNAc; (2) GlcNAcβ1→Asn; (3) GlcNAcβ1→2Man; (4) GlcNAcβ1→4Man; (5) GlcNAcβ1→6Man; (6) GlcNAcβ1→3Man; (7) GlcNAcα1→3Man; (8) GlcNAcβ1→.3Gal; (9) GlcNAcβ1→4Gal; (10) GlcNAcβ1→6Gal; (11 ) GlcNAcα1→4Gal; (12 ) GlcNAcα1→4GlcNAc; (13) GlcNAcβ1→6GalNAc; (14) GlcNAcβ1→3GalNAc; (15) GlcNAcβ→4GlcUA; (16) GlcNAcα1→4GlcUA; (17) GlcNAcα1→4IdUA. See, for example, U.S. Pat. Nos. 6,268,193; and 5,691,180.

7. Multiple-Enzyme Oligosaccharide Synthesis

As discussed above, in some embodiments, two or more enzymes are used to form a desired oligosaccharide moiety. For example, a particular oligosaccharide moiety might require addition of a galactose, a sialic acid, and a fucose in order to exhibit a desired activity. Accordingly, the invention provides methods in which two or more enzymes, e.g., glycosyltransferases, trans-sialidases, or sulfotransferases, are used to obtain high-yield synthesis of a desired oligosaccharide determinant.

In some cases, a substrate-linked oligosaccharide will include an acceptor moiety for the particular glycosyltransferase of interest upon in vivo biosynthesis of the substrate. Such substrates can be glycosylated using the methods of the invention without prior modification of the glycosylation pattern of the substrate. In other cases, however, a substrate of interest will lack a suitable acceptor moiety. In such cases, the methods of the invention can be used to alter the glycosylation pattern of the substrate so that the substrate-linked oligosaccharides then include an acceptor moiety for the glycosyltransferase-catalyzed attachment of a preselected saccharide unit of interest to form a desired oligosaccharide determinant.

Substrate-linked oligosaccharides optionally can be first “trimmed,” either in whole or in part, to expose either an acceptor moiety for the glycosyltransferase or a moiety to which one or more appropriate residues can be added to obtain a suitable acceptor. Enzymes such as glycosyltransferases and endoglycosidases are useful for the attaching and trimming reactions.

In an exemplary embodiment, the multiple enzyme methodology discussed in the preceding section leads to the formation of a saccharide that include a GalNAc, glucose, galactose, fucose and a sialic acid.

Either a sialyltransferase or a trans-sialidase (for α2,3-linked sialic acid only) can be used in these methods. The trans-sialidase reaction involves incubating the protein to be modified with a reaction mixture that contains a suitable amount of a galactosyltransferase (galβ1,3 or galβ1,4), a suitable galactosyl donor (e.g., UDP-galactose), a trans-sialidase, a suitable sialic acid donor substrate, a fucosyltransferase (capable of making an α1,3 or α1,4 linkage), a suitable fucosyl donor substrate (e.g., GDP-fucose), and a divalent metal ion. These reactions can be carried out either sequentially or simultaneously.

If a sialyltransferase is used, in an exemplary embodiment, the method involves incubating the protein to be modified with a reaction mixture that contains a suitable amount of a galactosyltransferase (galβ1,3 or galβ1,4), a suitable galactosyl donor (e.g., UDP-galactose), a sialyltransferase (α2,3 or α2,6) and a suitable sialic acid donor substrate (e.g., CMP sialic acid). The reaction is allowed to proceed substantially to completion, and then a fucosyltransferase (capable of making an α1,3 or α1,4 linkage) and a suitable fucosyl donor substrate (eg. GDP-fucose) are added. If a fucosyltransferase is used that requires a sialylated substrate (e.g., FucT VII), the reactions can be conducted simultaneously.

8. Glycosyltransferase Reaction Mixtures

The glycosyltransferases, substrates, and other reaction mixture ingredients described above are combined by admixture in an aqueous reaction medium (solution). The medium generally has a pH value of about 5 to about 9. The selection of a medium is based on the ability of the medium to maintain pH value at the desired level. Thus, in some embodiments, the medium is buffered to a pH value of about 7.5. If a buffer is not used, the pH of the medium should be maintained at about 5 to 8.5, depending upon the particular glycosyltransferase used. For fucosyltransferases, the pH range is preferably maintained from about 7.2 to 7.8. For sialyltransferases, the range is preferably from about 5.5 and about 6.5. A suitable base is NaOH, preferably 6 M NaOH.

Enzyme amounts or concentrations are expressed in activity Units, which is a measure of the initial rate of catalysis. One activity Unit catalyzes the formation of 1 μmol of product per minute at a given temperature (typically 37° C.) and pH value (typically 7.5). Thus, 10 Units of an enzyme is a catalytic amount of that enzyme where 10 μmol of substrate are converted to 10 μmol of product in one minute at a temperature of 37° C. and a pH value of 7.5.

The reaction medium may also comprise solubilizing detergents (e.g., Triton or SDS) and organic solvents, e.g., methanol or ethanol, if necessary. The enzymes can be utilized free in solution or can be bound to a support such as a polymer. The reaction mixture is thus substantially homogeneous at the beginning, although some precipitate can form during the reaction.

The temperature at which an above process is carried out can range from just above freezing to the temperature at which the most sensitive enzyme denatures. That temperature range is preferably about zero degrees C to about 45° C., and more preferably at about 20° C. to about 37° C.

The reaction mixture so formed is maintained for a period of time sufficient to obtain the desired high yield of desired oligosaccharide determinants present on oligosaccharide groups attached to the substrate to be glycosylated. For large-scale preparations, the reaction will often be allowed to proceed for about 8-240 hours, with a time of between about 12 and 72 hours being more typical.

In embodiments in which more than one glycosyltransferase is used to obtain the compositions of substrates having substantially uniform substrates, the enzymes and reagents for a second glycosyltransferase reaction can be added to the reaction medium once the first glycosyltransferase reaction has neared completion. For some combinations of enzymes, the glycosyltransferases and corresponding substrates can be combined in a single initial reaction mixture; the enzymes in such simultaneous reactions preferably do not form a product that cannot serve as an acceptor for the other enzyme. For example, most sialyltransferases do not sialylate a fucosylated acceptor, so unless a fucosyltransferase that only works on sialylated acceptors is used (e.g., FucT VII), a simultaneous reaction by both enzymes will most likely not result in the desired high yield of the desired oligosaccharide determinant. By conducting two glycosyltransferase reactions in sequence in a single vessel, overall yields are improved over procedures in which an intermediate species is isolated. Moreover, cleanup and disposal of extra solvents and by-products is reduced.

One or more of the glycosyltransferase reactions can be carried out as part of a glycosyltransferase cycle. Preferred conditions and descriptions of glycosyltransferase cycles have been described. A number of glycosyltransferase cycles (for example, sialyltransferase cycles, galactosyltransferase cycles, and fucosyltransferase cycles) are described in U.S. Pat. No. 5,374,541 and WO 9425615 A. Other glycosyltransferase cycles are described in Ichikawa et al. J. Am. Chem. Soc. 114:9283 (1992), Wong et al. J. Org. Chem. 57: 4343 (1992), DeLuca, et al., J. Am. Chem. Soc. 117:5869-5870 (1995), and Ichikawa et al. In Carbohydrates and Carbohydrate Polymers. Yaltami, ed. (ATL Press, 1993).

For the above glycosyltransferase cycles, the concentrations or amounts of the various reactants used in the processes depend upon numerous factors including reaction conditions such as temperature and pH value, and the choice and amount of acceptor saccharides to be glycosylated. Because the glycosylation process permits regeneration of activating nucleotides, activated donor sugars and scavenging of produced PPi in the presence of catalytic amounts of the enzymes, the process is limited by the concentrations or amounts of the stoichiometric substrates discussed before. The upper limit for the concentrations of reactants that can be used in accordance with the method of the present invention is determined by the solubility of such reactants.

Preferably, the concentrations of activating nucleotides, phosphate donor, the donor sugar and enzymes are selected such that glycosylation proceeds until the acceptor is consumed. The considerations discussed below, while in the context of a sialyltransferase, are generally applicable to other glycosyltransferase cycles.

Each of the enzymes is present in a catalytic amount. The catalytic amount of a particular enzyme varies according to the concentration of that enzyme's substrate as well as to reaction conditions such as temperature, time and pH value. Means for determining the catalytic amount for a given enzyme under preselected substrate concentrations and reaction conditions are well known to those of skill in the art.

In another exemplary embodiment the reaction mixture contains at least one glycosyl transferase, a donor substrate, an acceptor sugar and a divalent metal cation. The concentration of the divalent metal cation in the reaction medium is maintained between about 2 mM and about 75 mM, preferably between about 5 mM and about 50 mM and more preferably between about 5 and about 30 mM.

By periodically monitoring the metal ion concentration in the reaction medium and supplementing the medium by additional amounts of divalent metal ions, the reaction cycles can be driven to completion within a suitable timeframe. Additionally, if more than one glycosyltransferase is used, consecutive cycles can be carried out in the same reaction vessel without isolation of the intermediate product. Moreover, by removing the inhibitory pyrophosphate, the reaction cycles can be run at substantially higher substrate (acceptor) concentration. Preferred divalent metal ions for use in the present invention include Mn⁺⁺, Mg⁺, Co⁺⁺, Ca⁺, Zn⁺⁺ and combinations thereof. More preferably, the divalent metal ion is Mn⁺⁺.

In a further exemplary embodiment, the methods are carried out using a glycosyltransferase, e.g., sialyltransferase at a concentration of about 50 mU per mg of glycoprotein or less, preferably between about 5-25 mU per mg of glycoprotein. Typically, the concentration of sialyltransferase in the reaction mixture will be between about 10-50 mU/ml, with the glycoprotein concentration being at least about 2 mg/ml of reaction mixture. In a preferred embodiment, the method results in glycosylation, e.g., sialylation of greater than about 80% of the appropriate glycosyl acceptor moieties on the saccharide. Generally, the time required to obtain greater than about 80% glycosylation is less than or equal to about 48 hours.

9. Other Glycosyltransferases

Other glycosyltransferases can be substituted into similar transferase cycles as have been described in detail for the fucosyltransferases and sialyltransferases. In particular, the glycosyltransferase can also be, for instance, glucosyltransferases, e.g., Alg8 (Stagljov et al., Proc. Natl. Acad. Sci. USA 91:5977 (1994)) or Alg5 (Heesen et al. Eur. J. Biochem. 224:71 (1994)), N-acetylgalactosaminyltransferases such as, for example, α(1,3) N-acetylgalactosaminyltransferase, β(1,4) N-acetylgalactosaminyltransferases (Nagata et al. J. Biol. Chem. 267:12082-12089 (1992) and Smith et al. J. Biol Chem. 269:15162 (1994)) and polypeptide N-acetylgalactosaminyltransferase (Homa et al. J. Biol Chem. 268:12609 (1993)). Suitable N-acetylglucosaminyltransferases include GnTI (2.4.1.101, Hull et al., BBRC 176:608 (1991)), GnTII, and GnTIII (Ihara et al. J. Biochem. 113:692 (1993)), GnTV (Shoreiban et al. J. Biol. Chem. 268: 15381 (1993)), O-linked N-acetylglucosaminyltransferase (Bierhuizen et al. Proc. Natl. Acad. Sci. USA 89:9326 (1992)), N-acetylglucosamine-1-phosphate transferase (Rajput et al. Biochem J. 285:985 (1992), and hyaluronan synthase. Suitable mannosyltransferases include α(1,2) mannosyltransferase, α(1,3) mannosyltransferase, β(1,4) mannosyltransferase, Dol-P-Man synthase, OCh1, and Pmt1.

10. Purification

The products produced by the above processes can be used without purification. However, for some applications it is desirable to purify the substrates. Standard, well-known techniques for purification of substrates are suitable. Affinity chromatography is one example of a suitable purification method. A ligand that has affinity for a particular substrate or a particular oligosaccharide determinant on a substrate is attached to a chromatography matrix and the substrate composition is passed through the matrix. After an optional washing step, the substrate is eluted from the matrix.

Filtration can also be used for purification of substrates (see, e.g., U.S. Pat. Nos. 5,259,971 and 6,022,742.

If purification of the substrate is desired, it is preferable that the substrate be recovered in a substantially purified form. However, for some applications, no purification or only an intermediate level of purification of the substrate is required.

Moreover, according to another aspect of the invention, there is provided an improved method of purification of reaction products, such as those prepared according to the processes of the present invention, using membranes and organic solvent. Glycolipids and glycosphingolipids can be purified by this method of purification. Any of the enzyme reaction products described herein can be purified according to this method of purification. The method comprises concentrating a reaction product in a membrane purification system with the addition of an organic solvent. Suitable solvents include, but are not limited to alcohols (e.g., methanol), halocarbons (e.g., chloroform), and mixtures of hydrocarbons and alcohols (e.g., xylenes/methanol). In a preferred embodiment, the solvent is methanol. The concentration step can concentrate the reaction product to any selected degree. In an exemplary embodiment, the degree of concentration is from about 1- to about 100-fold, including from about 5- to about 50-fold, also including from about 10- to about 20-fold. The membrane purification system is selected from a variety of such systems known to those of skill in the art. In preferred embodiments, the membrane purification system is a 10K hollow fiber membrane purification system. In an exemplary embodiment, the method comprises concentrating the reaction mixture about ten-fold using a 10K hollow fiber membrane purification system, adding water and diafiltering the solution to about one-tenth the original volume, adding methanol to the retentate, and diafiltering to allow the reaction product to pass in the permeate. Concentration of the permeate solution yields the reaction product.

The products produced by the above processes can be used without purification. However, it is usually preferred to recover the product. Standard, well-known techniques for recovery of glycosylated saccharides such as thin or thick layer chromatography, column chromatography, ion exchange chromatography, or membrane filtration can be used. It is preferred to use membrane filtration, more preferably utilizing a reverse osmotic membrane, or one or more column chromatographic techniques for the recovery as is discussed hereinafter and in the literature cited herein. For instance, membrane filtration wherein the membranes have molecular weight cutoff of about 3000 to about 10,000 can be used to remove proteins such as glycosyl transferases. Nanofiltration or reverse osmosis can then be used to remove salts and/or purify the product saccharides (see, e.g., WO 98/15581). Nanofilter membranes are a class of reverse osmosis membranes that pass monovalent salts but retain polyvalent salts and uncharged solutes larger than about 100 to about 2,000 Daltons, depending upon the membrane used. Thus, in a typical application, saccharides prepared by the methods of the present invention will be retained in the membrane and contaminating salts will pass through.

The compounds prepared by a method of the invention may be separated from impurities by one or more steps selected from immunoaffinity chromatography, ion-exchange column fractionation (e.g., on diethylaminoethyl (DEAE) or matrices containing carboxymethyl or sulfopropyl groups), chromatography on Blue-Sepharose, CM Blue-Sepharose, MONO-Q, MONO-S, lentil lectin-Sepharose, WGA-Sepharose, Con A-Sepharose, Ether Toyopearl, Butyl Toyopearl, Phenyl Toyopearl, or protein A Sepharose, SDS-PAGE chromatography, silica chromatography, chromatofocusing, reverse phase HPLC (e.g., silica gel with appended aliphatic groups), gel filtration using, e.g., Sephadex molecular sieve or size-exclusion chromatography, and chromatography on columns that selectively bind compound.

Within another embodiment, supernatants from systems which produce a compound by the method of the invention are first concentrated using a commercially available protein concentration filter, for example, an Amicon or Millipore Pellicon ultrafiltration unit. Following the concentration step, the concentrate may be applied to a suitable purification matrix. For example, a suitable affinity matrix may comprise a ligand for the glycolipid bound to a suitable support. Alternatively, an anion-exchange resin may be employed, for example, a matrix or substrate having pendant DEAE groups. Suitable matrices include acrylamide, agarose, dextran, cellulose, or other types commonly employed in protein purification. Alternatively, a cation-exchange step may be employed. Suitable cation exchangers include various insoluble matrices comprising sulfopropyl or carboxymethyl groups. Sulfopropyl groups are particularly preferred.

Finally, one or more RP-HPLC steps employing hydrophobic RP-HPLC media, e.g., silica gel having pendant methyl or other aliphatic groups, may be employed to further purify a polypeptide variant composition. Some or all of the foregoing purification steps, in various combinations, can also be employed to provide a homogeneous modified glycoprotein.

The glycolipid of the invention resulting from a large-scale fermentation may be purified by methods analogous to those disclosed by Urdal et al., J. Chromatog. 296: 171 (1984). This reference describes two sequential, RP-HPLC steps for purification of recombinant human IL-2 on a preparative HPLC column. Alternatively, techniques such as affinity chromatography may be utilized to purify the modified glycolipid.

Conjugation

The compounds produced by method of the invention, in their unconjugated form are generally useful as therapeutic agents. The compounds of the invention can be conjugated to a wide variety of compounds to create specific labels, probes, separation media, diagnostic and/or therapeutic reagents, etc. Examples of species to which the compounds of the invention can be conjugated include, for example, biomolecules such as proteins (e.g., antibodies, enzymes, receptors, etc.), nucleic acids (e.g., RNA, DNA, etc.), bioactive molecules (e.g., drugs, toxins, etc.), detectable labels (e.g., fluorophores, radioactive isotopes), solid substrates such as glass or polymeric beads, sheets, fibers, membranes (e.g. nylon, nitrocellulose), slides (e.g. glass, quartz) and probes; etc.

Linkers

The compounds of the invention can be functionalized with one or more linker moieties, linking the compound to a group, through which the compound may optionally be tethered to another species. The linker can be appended to a glycosyl moiety (e.g., sialic acid), which, in spite of the modification, the serves as a substrate for an appropriate glycosyltransferase.

Preparation of the modified sugar for use in the methods of the present invention includes attachment of a modifying group to a sugar residue and forming a stable adduct, which is a substrate for a glycosyltransferase. Thus, it is often preferred to use a cross-linking agent to conjugate the modifying group and the sugar. Exemplary bifunctional compounds which can be used for attaching modifying groups to carbohydrate moieties include, but are not limited to, bifunctional poly(ethyleneglycols), polyamides, polyethers, polyesters and the like. General approaches for linking carbohydrates to other molecules are known in the literature. See, for example, Lee et al., Biochemistry 28: 1856 (1989); Bhatia et al., Anal. Biochem. 178: 408 (1989); Janda et al., J. Am. Chem. Soc. 112: 8886 (1990) and Bednarski et al., WO 92/18135. In the discussion that follows, the reactive groups are treated as benign on the sugar moiety of the nascent modified sugar. The focus of the discussion is for clarity of illustration. Those of skill in the art will appreciate that the discussion is relevant to reactive groups on the modifying group as well.

An exemplary strategy involves incorporation of a protected sulfhydryl onto the sugar using the heterobifunctional crosslinker SPDP (n-succinimidyl-3-(2-pyridyldithio)propionate and then deprotecting the sulfhydryl for formation of a disulfide bond with another sulfhydryl on the modifying group.

If SPDP detrimentally affects the ability of the modified sugar to act as a glycosyltransferase substrate, one of an array of other crosslinkers such as 2-iminothiolane or N-succinimidyl S-acetylthioacetate (SATA) is used to form a disulfide bond. 2-iminothiolane reacts with primary amines, instantly incorporating an unprotected sulfhydryl onto the amine-containing molecule. SATA also reacts with primary amines, but incorporates a protected sulfhydryl, which is later deacetaylated using hydroxylamine to produce a free sulfhydryl. In each case, the incorporated sulfhydryl is free to react with other sulfhydryls or protected sulfhydryl, like SPDP, forming the required disulfide bond.

The above-described strategy is exemplary, and not limiting, of linkers of use in the invention. Other crosslinkers are available that can be used in different strategies for crosslinking the modifying group to the peptide. For example, TPCH(S-(2-thiopyridyl)-L-cysteine hydrazide and TPMPH ((S-(2-thiopyridyl) mercapto-propionohydrazide) react with carbohydrate moieties that have been previously oxidized by mild periodate treatment, thus forming a hydrazone bond between the hydrazide portion of the crosslinker and the periodate generated aldehydes. TPCH and TPMPH introduce a 2-pyridylthione protected sulfhydryl group onto the sugar, which can be deprotected with DTT and then subsequently used for conjugation, such as forming disulfide bonds between components.

If disulfide bonding is found unsuitable for producing stable modified sugars, other crosslinkers may be used that incorporate more stable bonds between components. The heterobifunctional crosslinkers GMBS (N-gama-malimidobutyryloxy)succinimide) and SMCC (succinimidyl 4-(N-maleimido-methyl)cyclohexane) react with primary amines, thus introducing a maleimide group onto the component. The maleimide group can subsequently react with sulfhydryls on the other component, which can be introduced by previously mentioned crosslinkers, thus forming a stable thioether bond between the components. If steric hindrance between components interferes with either component's activity or the ability of the modified sugar to act as a glycosyltransferase substrate, crosslinkers can be used which introduce long spacer arms between components and include derivatives of some of the previously mentioned crosslinkers (i.e., SPDP). Thus, there is an abundance of suitable crosslinkers, which are useful; each of which is selected depending on the effects it has on optimal peptide conjugate and modified sugar production.

In another exemplary embodiment, the lipid is converted to the corresponding aldehydes or ketone (e.g., by ozonization) and an amine containing carrier molecule is derivatized via reductive amination with the modified lipid.

A variety of reagents are used to modify the components of the modified sugar with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (Holcenberg, and Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

In an exemplary embodiment, the invention provides a compound according to Formula I, wherein a member selected from a glycosyl residue or Y has the formula: 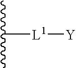
in which L¹ is a member selected from substituted or unsubstituted alkyl, substituted or unsubstituted heteroalkyl and substituted or unsubstituted aryl; and Y is a member selected from protected or unprotected reactive functional groups, detectable labels and targeting moieties.

In another exemplary embodiment, L¹ is an ether or a polyether, preferably a member selected from ethylene glycol, ethylene glycol oligomers and combinations thereof, having a molecular weight of from about 60 daltons to about 10,000 daltons, and more preferably of from about 100 daltons to about 1,000 daltons.

Representative polyether-based substituents include, but are not limited to, the following structures: 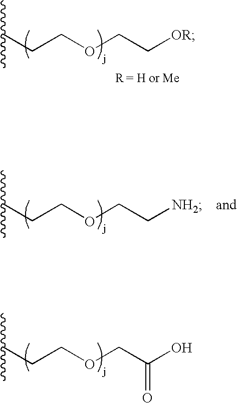
in which j is preferably a number from 1 to 100, inclusive. Other functionalized polyethers are known to those of skill in the art, and many are commercially available from, for example, Shearwater Polymers, Inc. (Alabama).

In another preferred embodiment, the linker includes a reactive group for conjugating the oligosaccharide compound to a molecule or a surface. Representative useful reactive groups are discussed in greater detail in the succeeding section. Additional information on useful reactive groups is known to those of skill in the art. See, for example, Hermanson, BIOCONJUGATE TECHNIQUES, Academic Press, San Diego, 1996.

Modified glycosyl donor species (“modified sugars”) are preferably selected from modified sugar nucleotides, activated modified sugars and modified sugars that are simple saccharides that are neither nucleotides nor activated. Any desired carbohydrate structure can be added to a substrate using the methods of the invention. Typically, the structure will be a monosaccharide, but the present invention is not limited to the use of modified monosaccharide sugars; oligosaccharides and polysaccharides are useful as well.

The modifying group is attached to a sugar moiety by enzymatic means, chemical means or a combination thereof, thereby producing a modified sugar. The sugars are substituted at any position that allows for the attachment of the modifying moiety, yet which still allows the sugar to function as a substrate for the enzyme used to ligate the modified sugar to the substrate. In a preferred embodiment, when sialic acid is the sugar, the sialic acid is substituted with the modifying group at either the 9-position on the pyruvyl side chain or at the 5-position on the amine moiety that is normally acetylated in sialic acid.

In certain embodiments of the present invention, a modified sugar nucleotide is utilized to add the modified sugar to the substrate. Exemplary sugar nucleotides that are used in the present invention in their modified form include nucleotide mono-, di- or triphosphates or analogs thereof. In a preferred embodiment, the modified sugar nucleotide is selected from a UDP-glycoside, CMP-glycoside, or a GDP-glycoside. Even more preferably, the modified sugar nucleotide is selected from an UDP-galactose, UDP-galactosamine, UDP-glucose, UDP-glucosamine, GDP-mannose, GDP-fucose, CMP-sialic acid, or CMP-NeuAc.

The invention also provides methods for synthesizing a compound using a modified sugar, e.g., modified-galactose, -fucose, and -sialic acid. When a modified sialic acid is used, either a sialyltransferase or a trans-sialidase (for α2,3-linked sialic acid only) can be used in these methods.

In other embodiments, the modified sugar is an activated sugar. Activated modified sugars, which are useful in the present invention are typically glycosides which have been synthetically altered to include an activated leaving group. As used herein, the term “activated leaving group” refers to those moieties, which are easily displaced in enzyme-regulated nucleophilic substitution reactions. Many activated sugars are known in the art. See, for example, Vocadlo et al., In CARBOHYDRATE CHEMISTRY AND BIOLOGY, Vol. 2, Ernst et al. Ed., Wiley-VCH Verlag: Weinheim, Germany, 2000; Kodama et al., Tetrahedron Lett. 34: 6419 (1993); Lougheed, et al., J. Biol. Chem. 274: 37717 (1999)).

Examples of activating groups include fluoro, chloro, bromo, tosylate ester, mesylate ester, triflate ester and the like. Preferred activated leaving groups, for use in the present invention, are those that do not significantly sterically encumber the enzymatic transfer of the glycoside to the acceptor. Accordingly, preferred embodiments of activated glycoside derivatives include glycosyl fluorides and glycosyl mesylates, with glycosyl fluorides being particularly preferred. Among the glycosyl fluorides, α-galactosyl fluoride, α-mannosyl fluoride, α-glucosyl fluoride, α-fucosyl fluoride, α-xylosyl fluoride, α-sialyl fluoride, α-N-acetylglucosaminyl fluoride, α-N-acetylgalactosaminyl fluoride, β-galactosyl fluoride, β-mannosyl fluoride, β-glucosyl fluoride, β-fucosyl fluoride, β-xylosyl fluoride, β-sialyl fluoride, β-N-acetylglucosaminyl fluoride and β-N-acetylgalactosaminyl fluoride are most preferred.

By way of illustration, glycosyl fluorides can be prepared from the free sugar by first acetylating the sugar and then treating it with HF/pyridine. This generates the thermodynamically most stable anomer of the protected (acetylated) glycosyl fluoride (i.e., the α-glycosyl fluoride). If the less stable anomer (i.e., the β-glycosyl fluoride) is desired, it can be prepared by converting the peracetylated sugar with HBr/HOAc or with HCI to generate the anomeric bromide or chloride. This intermediate is reacted with a fluoride salt such as silver fluoride to generate the glycosyl fluoride. Acetylated glycosyl fluorides may be deprotected by reaction with mild (catalytic) base in methanol (e.g. NaOMe/MeOH). In addition, many glycosyl fluorides are commercially available.

Other activated glycosyl derivatives can be prepared using conventional methods known to those of skill in the art. For example, glycosyl mesylates can be prepared by treatment of the fully benzylated hemiacetal form of the sugar with mesyl chloride, followed by catalytic hydrogenation to remove the benzyl groups.

In a further exemplary embodiment, the modified sugar is an oligosaccharide having an antennary structure. In a preferred embodiment, one or more of the termini of the antennae bear the modifying moiety. When more than one modifying moiety is attached to an oligosaccharide having an antennary structure, the oligosaccharide is useful to “amplify” the modifying moiety; each oligosaccharide unit conjugated to the peptide attaches multiple copies of the modifying group to the peptide.

Reactive Functional Groups

As discussed above, certain of the compounds of the invention bear a reactive functional group, such as a component of a linker arm, which can be located at any position on any aryl nucleus or on a chain, such as an alkyl chain, attached to an aryl nucleus, or on the backbone of the chelating agent. These compounds are referred to herein as “reactive ligands.” When the reactive group is attached to an alkyl, or substituted alkyl chain tethered to an aryl nucleus, the reactive group is preferably located at a terminal position of an alkyl chain. Reactive groups and classes of reactions useful in practicing the present invention are generally those that are well known in the art of bioconjugate chemistry. Currently favored classes of reactions available with reactive ligands of the invention are those, which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

The reactive functional groups can be chosen such that they do not participate in, or interfere with, the reactions necessary to assemble the oligosaccharide. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

Detectable Labels

In an exemplary embodiment, the compound prepared by a method of the invention includes a detectable label, such as a fluorophores or radioactive isotope. The detectable label can be appended to a glycosyl moiety (e.g., sialic acid) by means of a linker arm in a manner that still allows the labeled glycosyl moiety serves as a substrate for an appropriate glycosyltransferase as discussed herein.

The embodiment of the invention in which a label is utilized is exemplified by the use of a fluorescent label. Fluorescent labels have the advantage of requiring few precautions in their handling, and being amenable to high-throughput visualization techniques (optical analysis including digitization of the image for analysis in an integrated system comprising a computer). Preferred labels are typically characterized by high sensitivity, high stability, low background, long lifetimes, low environmental sensitivity and high specificity in labeling.

Many fluorescent labels can be incorporated into the compositions of the invention. Many such labels are commercially available from, for example, the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill. Furthermore, those of skill in the art will recognize how to select an appropriate fluorophore for a particular application and, if it not readily available commercially, will be able to synthesize the necessary fluorophore de novo or synthetically modify commercially available fluorescent compounds to arrive at the desired fluorescent label.

Polymers

In another exemplary embodiment, the invention provides a polymer that includes a subunit according to Formula I. The polymer may be a synthetic polymer (e.g., poly(styrene), poly(acrylamide), poly(lysine), polyethers, polyimines, dendrimers, cyclodextrins, and dextran) or a biopolymer, e.g, polypeptides (e.g., antibody, enzyme, serum protein), saccharide, nucleic acid, antigen, hapten, etc. The polymer may have an activity associated with it (e.g., an antibody) or it may simply serve as a carrier molecule (e.g., a dendrimer).

The carrier molecules may also be used as a backbone for compounds of the invention that are poly- or multi-valent species, including, for example, species such as dimers, trimers, tetramers and higher homologs of the compounds of the invention or reactive analogues thereof. The poly- and multi-valent species can be assembled from a single species or more than one species of the invention. For example, a dimeric construct can be “homo-dimeric” or “heterodimeric.” Moreover, poly- and multi-valent constructs in which a compound of the invention or a reactive analogue thereof, is attached to an oligomeric or polymeric framework (e.g., polylysine, dextran, hydroxyethyl starch and the like) are within the scope of the present invention. The framework is preferably polyfunctional (i.e. having an array of reactive sites for attaching compounds of the invention). Moreover, the framework can be derivatized with a single species of the invention or more than one species of the invention.

Moreover, the properties of the carrier molecule can be selected to afford compounds having water-solubility that is enhanced relative to analogous compounds that are not similarly functionalized. Thus, any of the substituents set forth herein can be replaced with analogous radicals that have enhanced water solubility. For example, it is within the scope of the invention to, for example, replace a hydroxyl group with a diol, or an amine with a quaternary amine, hydroxylamine or similar more water-soluble moiety. In a preferred embodiment, additional water solubility is imparted by substitution at a site not essential for the activity towards the ion channel of the compounds set forth herein with a moiety that enhances the water solubility of the parent compounds. Methods of enhancing the water-solubility of organic compounds are known in the art. Such methods include, but are not limited to, functionalizing an organic nucleus with a permanently charged moiety, e.g., quaternary ammonium, or a group that is charged at a physiologically relevant pH, e.g. carboxylic acid, amine. Other methods include, appending to the organic nucleus hydroxyl- or amine-containing groups, e.g. alcohols, polyols, polyethers, and the like. Representative examples include, but are not limited to, polylysine, polyethyleneimine, poly(ethyleneglycol) and poly(propyleneglycol). Suitable functionalization chemistries and strategies for these compounds are known in the art. See, for example, Dunn, R. L., et al., Eds. POLYMERIC DRUGS AND DRUG DELIVERY SYSTEMS, ACS Symposium Series Vol. 469, American Chemical Society, Washington, D.C. 1991.

In another embodiment, the compound produced by the method of the invention is attached to an immunogenic carrier. Commonly used carriers are large molecules that are highly immunogenic and capable of imparting their immunogenicity to a hapten coupled to the carrier. Examples of carriers include, but are not limited to, proteins, lipid bilayers (e.g., liposomes), synthetic or natural polymers (e.g., dextran, agarose, poly-L-lysine) or synthetic organic molecules. Preferred immunogenic carriers are those that are immunogenic, have accessible functional groups for conjugation with a hapten, are reasonably water-soluble after derivitization with a hapten, and are substantially non-toxic in vivo. Presently preferred carriers include, for example protein carriers having a molecular weight of greater than or equal to 5000 daltons, more preferably, albumin or hemocyanin.

The immunogenicity of compositions prepared by the methods of the present invention may further be enhanced by linking the composition to-one or more peptide sequences that are able to a elicit a cellular immune response (see, e.g., WO 94/20127). Peptides that stimulate cytotoxic T lymphocyte (CTL) responses as well as peptides that stimulate helper T lymphocyte (HTL) responses are useful for linkage to the compounds of the invention. The peptides can be linked by a linker moiety as discussed above. An exemplary linker is typically comprised of relatively small, neutral molecules, such as amino acids or amino acid mimetics, which are uncharged under physiological conditions.

A compound prepared by a method of the invention may be linked to a T helper peptide that is recognized by T helper cells in the majority of the population. This can be accomplished by selecting amino acid sequences that bind to many, most, or all of the HLA class II molecules. An example of such a T helper peptide is tetanus toxoid at positions 830-843 (see, e.g., Panina-Bordignon et al., Eur. J. Immunol. 19: 2237-2242 (1989)).

Further, a compound prepared by a method of the invention may be linked to multiple antigenic determinants to enhance immunogenicity. For example, in order to elicit recognition by T cells of multiple HLA types, a synthetic peptide encoding multiple overlapping T cell antigenic determinants (cluster peptides) may be used to enhance immunogenicity (see, e.g., Ahlers et al., J. Immunol. 150: 5647-5665 (1993)). Such cluster peptides contain overlapping, but distinct antigenic determinants. The cluster peptide may be synthesized colinearly with a peptide of the invention. The cluster peptide may be linked to a compound of the invention by one or more spacer molecules.

A peptide composition comprising a compound of the invention linked to a cluster peptide may also be used in conjunction with a cluster peptide linked to a CTL-inducting epitope. Such compositions may be administered via alternate routes or using different adjuvants.

Alternatively multiple peptides encoding CTL and/or HTL epitopes may be used in conjunction with a compound of the invention.

Many methods are known to those of skill in the art for coupling a hapten to a carrier. In an exemplary embodiment, a glycolipid prepared by the method of the invention includes a sulfhydryl group that is readily combined with keyhole limpet hemocyanin, which has been activated by SMCC (succinimidyl-4-(N-maleimidomethyl)cyclohexane-1-carboxylate), Dewey et al., Proc. Natl. Acad. Sci. USA 84: 53745378 (1987). The sulfhydryl-bearing lipid useful in this method can be synthesized by a number of art-recognized methods. For example, a lipid bearing a terminal carboxyl group is coupled with cysteamine, using a dehydrating agent, such as dicyclohexylcarbodiimide (DCC), to form a dimeric glycolipid, linked via a disulfide bridge. The disulfide bridge is cleaved by reduction, affording the monomeric sulfhydryl-derivatized glycolipid.

In yet another preferred embodiment, the composition includes a linker moiety situated between the glycolipid and the carrier. The discussion above regarding the characteristics of linker moieties is substantially applicable to the present embodiment. In an exemplary embodiment, the linker arm includes a poly(ethyleneglycol) (PEG) group. Bifunctional PEG derivative appropriate for use in this method are commercially available (Shearwater Polymers) or can be prepared by methods well known in the art. In an exemplary embodiment, the SMCC activated KLH, infra, is reacted with a PEG-glycolipid conjugate, bearing a sulfhydryl group. An appropriate conjugate can be prepared by a number of synthetic routes accessible to those of skill in the art. For example, a commercially available product, such as t-Boc-NH-PEG-NH₂, is reacted with a carboxyl terminal glycolipid in the presence of a dehydrating agent (e.g., DCC), thereby forming the PEG amide of the glycolipid. The t-Boc group is removed by acid treatment (e.g., trifluoroacetic acid, TFA), to afford the deprotected amino PEG amide of the glycolipid. The deprotected glycolipid is subsequently reacted with a sulfhydryl protected molecule, such as 3-mercaptopropionic acid or a commercially available thiol and amine protected cysteine, in the presence of a dehydrating agent. The thiol group is then deprotected and the conjugate is reacted with the SMCC activated KLH to provide an autoinducer analogue linked to a carrier via a PEG spacer group.

The exemplary embodiments presented above are intended to illustrate general reaction schemes that are useful in preparing certain of the compounds of the present invention and should not be interpreted as limiting the scope of the invention or the pathways useful to produce the compounds of the invention.

Targeting Moieites

In addition to providing a polymeric “support” or backbone for TIAM and other cheating agents, carrier molecules can be used to target ligands (or complexes) of the invention to a specific region within the body or tissue, or to a selected species or structure in vitro. Selective targeting of an agent by its attachment to a species with an affinity for the targeted region is well known in the art. Both small molecule and polymeric targeting agents are of use in the present invention.

The ligands (or complexes) can be linked to targeting agents that selectively deliver it to a cell, organ or region of the body. Exemplary targeting agents such as antibodies, ligands for receptors, lectins, saccharides, antibodies, and the like are recognized in the art and are useful without limitation in practicing the present invention. Other targeting agents include a class of compounds that do not include specific molecular recognition motifs include macromolecules such as poly(ethylene glycol), polysaccharide, polyamino acids and the like, which add molecular mass to the ligand. The ligand-targeting agent conjugates of the invention are exemplified by the use of a nucleic acid-ligand conjugate. The focus on ligand-oligonucleotide conjugates is for clarity of illustration and is not limiting of the scope of targeting agents to which the ligands (or complexes) of the invention can be conjugated. Moreover, it is understood that “ligand” refers to both the free ligand and its metal complexes.

Exemplary nucleic acid targeting agents include aptamers, antisense compounds, and nucleic acids that form triple helices. Typically, a hydroxyl group of a sugar residue, an amino group from a base residue, or a phosphate oxygen of the nucleotide is utilized as the needed chemical functionality to couple the nucleotide-based targeting agent to the ligand. However, one of skill in the art will readily appreciate that other “non-natural” reactive functionalities can be appended to a nucleic acid by conventional techniques. For example, the hydroxyl group of the sugar residue can be converted to a mercapto or amino group using techniques well known in the art.

Aptamers (or nucleic acid antibody) are single- or double-stranded DNA or single-stranded RNA molecules that bind specific molecular targets. Generally, aptamers function by inhibiting the actions of the molecular target, e.g., proteins, by binding to the pool of the target circulating in the blood. Aptamers possess chemical functionality and thus, can covalently bond to ligands, as described herein.

Although a wide variety of molecular targets are capable of forming non-covalent but specific associations with aptamers, including small molecules drugs, metabolites, cofactors, toxins, saccharide-based drugs, nucleotide-based drugs, glycoproteins, and the like, generally the molecular target will comprise a protein or peptide, including serum proteins, kinins, eicosanoids, cell surface molecules, and the like. Examples of aptamers include Gilead's antithrombin inhibitor GS 522 and its derivatives (Gilead Science, Foster City, Calif). See also, Macaya et al. Proc. Natl. Acad. Sci. USA 90:3745-9 (1993); Bock et al. Nature (London) 355:564-566 (1992) and Wang et al. Biochem. 32:1899-904 (1993).

Aptamers specific for a given biomolecule can be identified using techniques known in the art. See, e.g., Toole et al. (1992) PCT Publication No. WO 92/14843; Tuerk and Gold (1991) PCT Publication No. WO 91/19813; Weintraub and Hutchinson (1992) PCT Publication No. 92/05285; and Ellington and Szostak, Nature 346:818 (1990). Briefly, these techniques typically involve the complexation of the molecular target with a random mixture of oligonucleotides. The aptamer-molecular target complex is separated from the uncomplexed oligonucleotides. The aptamer is recovered from the separated complex and amplified. This cycle is repeated to identify those aptamer sequences with the highest affinity for the molecular target.

Cleaveable Groups

The invention also provides methods of preparing oligosaccharide conjugates that are linked to another moiety (e.g., polymer, targeting moiety, detectable label, solid support) via a linkage that is designed to cleave, releasing the saccharide conjugate. Cleaveable groups include bonds that are reversible (e.g., easily hydrolyzed) or partially reversible (e.g., partially or slowly hydrolyzed). Cleavage of the bond can occur through biological or physiological processes. In other embodiments, the physiological processes cleave bonds at other locations within the complex (e.g., removing an ester group or other protecting group that is coupled to an otherwise sensitive chemical functionality) before cleaving the bond between the agent and dendrimer, resulting in partially degraded complexes. Other cleavages can also occur, for example, between a spacer and targeting agent and the spacer and the ligand.

In an exemplary embodiment, the linkage used in the method of the invention is degraded by enzymes such as non-specific aminopeptidases and esterases, dipeptidyl carboxypeptidases, proteases of the blood clotting cascade, and the like.

Alternatively, cleavage is through a nonenzymatic process. For example, chemical hydrolysis may be initiated by differences in pH experienced by the complex. In such a case, the complex may be characterized by a high degree of chemical lability at physiological pH of 7.4, while exhibiting higher stability at an acidic or basic pH in the delivery vehicle. An exemplary complex, which is cleaved in such a process is a complex incorporating a N-Mannich base linkage within its framework.

Another exemplary group of cleaveable compounds are those based on non-covalent protein binding groups discussed herein.

The susceptibility of the cleaveable group to degradation can be ascertained through studies of the hydrolytic or enzymatic conversion of the group. Generally, good correlation between in vitro and in vivo activity is found using this method. See, e.g., Phipps et al., J. Pharm. Sciences 78:365 (1989). The rates of conversion are readily determined, for example, by spectrophotometric methods or by gas-liquid or high-pressure liquid chromatography. Half-lives and other kinetic parameters may then be calculated using standard techniques. See, e.g., Lowry et al. MECHANISM AND THEORY IN ORGANIC CHEMISTRY, 2nd Ed., Harper & Row, Publishers, New York (1981).

The Compositions

In some embodiments, the invention provides a composition that has a substantially uniform glycosylation pattern. The compositions include a saccharide or oligosaccharide that is attached to a substrate for which a selected glycoform is desired. The composition is prepared by a method of the invention.

In the compositions of the invention, a preselected saccharide unit is linked to at least about 60% of the potential acceptor moieties of interest. More preferably, the preselected saccharide unit is linked to at least about 80% of the potential acceptor moieties of interest, and still more preferably to at least 95% of the potential acceptor moieties of interest. In situations in which the starting substrate exhibits heterogeneity in the oligosaccharide structure of interest (e.g., some of the oligosaccharides on the starting substrate already have the preselected saccharide unit attached to the acceptor moiety of interest), the recited percentages include such pre-attached saccharide units.

Pharmaceutical Formulations

In yet another embodiment, the invention provides a pharmaceutical formulation that includes a compound produced by a method according to the invention in admixture with a pharmaceutically acceptable carrier.

The substrates having desired oligosaccharide determinants described above can then be used in a variety of applications, e.g., as antigens, diagnostic reagents, or as therapeutics. Thus, the present invention also provides pharmaceutical compositions, which can be used in treating a variety of conditions. The pharmaceutical compositions are comprised of substrates made according to the methods described above.

Pharmaceutical compositions of the invention are suitable for use in a variety of drug delivery systems. Suitable formulations for use in the present invention are found in Remington 's Pharmaceutical Sciences, Mace Publishing Company, Philadelphia, Pa., 17th ed. (1985). For a brief review of methods for drug delivery, see, Langer, Science 249: 1527-1533 (1990).

The pharmaceutical compositions are intended for parenteral, intranasal, topical, oral or local administration, such as by aerosol or transdermally, for prophylactic and/or therapeutic treatment. Commonly, the pharmaceutical compositions are administered parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils, intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. The compositions may contain pharmaceutically acceptable auxiliary substances as required to approximate physiological conditions, such as pH adjusting and buffering agents, tonicity adjusting agents, wetting agents, detergents and the like.

The composition may also contain aglycolipid prepared by a method of the invention that is conjugated to an immunogenic species, e.g., KLH. Moreover, the compositions prepared by methods of the invention and their immunogenic conjugates may be combined with an adjuvant.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

The compositions containing the compounds can be administered for prophylactic and/or therapeutic treatments. In therapeutic applications, compositions are administered to a patient already suffering from a disease, as described above, in an amount sufficient to cure or at least partially arrest the symptoms of the disease and its complications. An amount adequate to accomplish this is defined as a “therapeutically effective dose.” Amounts effective for this use will depend on the severity of the disease and the weight and general state of the patient, but generally range from about 0.5 mg to about 2,000 mg of substrate per day for a 70 kg patient, with dosages of from about 5 mg to about 200 mg of the compounds per day being more commonly used.

In prophylactic applications, compositions containing the substrates of the invention are administered to a patient susceptible to or otherwise at risk of a particular disease. Such an amount is defined to be a “prophylactically effective dose.” In this use, the precise amounts again depend on the patient's state of health and weight, but generally range from about 0.5 mg to about 1,000 mg per 70 kilogram patient, more commonly from about 5 mg to about 200 mg per 70 kg of body weight.

Single or multiple administrations of the compositions can be carried out with dose levels and pattern being selected by the treating physician. In any event, the pharmaceutical formulations should provide a quantity of the substrates of this invention sufficient to effectively treat the patient.

The substrates can also find use as diagnostic reagents. For example, labeled substrates can be used to determine the locations at which the substrate becomes concentrated in the body due to interactions between the desired oligosaccharide determinant and the corresponding ligand. For this use, the compounds can be labeled with appropriate radioisotopes, for example, ¹²⁵I, ¹⁴C, or tritium, or with other labels known to those of skill in the art.

The dosage ranges for the administration of the gangliosides of the invention are those large enough to produce the desired effect in which the symptoms of the immune response show some degree of suppression. The dosage should not be so large as to cause adverse side effects. Generally, the dosage will vary with the age, condition, sex and extent of the disease in the animal and can be determined by one of skill in the art. The dosage can be adjusted by the individual physician in the event of any counterindications.

Additional pharmaceutical methods may be employed to control the duration of action. Controlled release preparations may be achieved by the use of polymers to conjugate, complex or adsorb the ganglioside. The controlled delivery may be exercised by selecting appropriate macromolecules (for example, polyesters, polyamino carboxymethylcellulose, and protamine sulfate) and the concentration of macromolecules as well as the methods of incorporation in order to control release. Another possible method to control the duration of action by controlled release preparations is to incorporate the ganglioside into particles of a polymeric material such as polyesters, polyamino acids, hydrogels, poly (lactic acid) or ethylene vinylacetate copolymers.

In order to protect the gangliosides from binding with plasma proteins, it is preferred that the gangliosides be entrapped in microcapsules prepared, for example, by coacervation techniques or by interfacial polymerization, for example, hydroxymethylcellulose or gelatin-microcapsules and poly (methymethacrylate) microcapsules, respectively, or in colloidal drug delivery systems, for example, liposomes, albumin microspheres, microemulsions, nanoparticles, and nanocapsules or in macroemulsions. Such teachings are disclosed in Remington's Pharmaceutical Sciences (16th Ed., A. Oslo, ed., Mack, Easton, Pa., 1980).

The gangliosides of the invention are well suited for use in targetable drug delivery systems such as synthetic or natural polymers in the form of macromolecular complexes, nanocapsules, microspheres, or beads, and lipid-based systems including oil-in-water emulsions, micelles, mixed micelles, liposomes, and resealed erythrocytes. These systems are known collectively as colloidal drug delivery systems. Typically, such colloidal particles containing the dispersed gangliosides are about 50 nm-2 μm in diameter. The size of the colloidal particles allows them to be administered intravenously such as by injection, or as an aerosol. Materials used in the preparation of colloidal systems are typically sterilizable via filter sterilization, nontoxic, and biodegradable, for example albumin, ethylcellulose, casein, gelatin, lecithin, phospholipids, and soybean oil. Polymeric colloidal systems are prepared by a process similar to the coacervation of microencapsulation.

In an exemplary embodiment, the gangliosides are components of a liposome, used as a targeted delivery system. When phospholipids are gently dispersed in aqueous media, they swell, hydrate, and spontaneously form multilamellar concentric bilayer vesicles with layers of aqueous media separating the lipid bilayer. Such systems are usually referred to as multilamellar liposomes or multilamellar vesicles (MLVs) and have diameters ranging from about 100 nm to about 4 μm. When MLVs are sonicated, small unilamellar vesicles (SUVS) with diameters in the range of from about 20 to about 50 nm are formed, which contain an aqueous solution in the core of the SUV.

Examples of lipids useful in liposome production include phosphatidyl compounds, such as phosphatidylglycerol, phosphatidylcholine, phosphatidylserine, and phosphatidylethanolamine. Particularly useful are diacylphosphatidylglycerols, where the lipid moiety contains from 14-18 carbon atoms, particularly from 16-18 carbon atoms, and are saturated. Illustrative phospholipids include egg phosphatidylcholine, dipalmitoylphosphatidylcholine, and distearoylphosphatidylcholine.

In preparing liposomes containing the gangliosides of the invention, such variables as the efficiency of ganglioside encapsulation, lability of the ganglioside, homogeneity and size of the resulting population of liposomes, ganglioside-to-lipid ratio, permeability instability of the preparation, and pharmaceutical acceptability of the formulation should be considered. Szoka, et al, Annual Review of Biophysics and Bioengineering, 9: 467 (1980); Deamer, et al., in LIPOSOMES, Marcel Dekker, New York, 1983, 27: Hope, et al., Chem. Phys. Lipids, 40: 89 (1986)).

The targeted delivery system containing the gangliosides of the invention may be administered in a variety of ways to a host, particularly a mammalian host, such as intravenously, intramuscularly, subcutaneously, intra-peritoneally, intravascularly, topically, intracavitarily, transdermally, intranasally, and by inhalation. The concentration of the gangliosides will vary upon the particular application, the nature of the disease, the frequency of administration, or the like. The targeted delivery system-encapsulated ganglioside may be provided in a formulation comprising other compounds as appropriate and an aqueous physiologically acceptable medium, for example, saline, phosphate buffered saline, or the like.

The compounds produced by a method of the invention can also be used as an immunogen for the production of monoclonal or polyclonal antibodies specifically reactive with the compounds of the invention. The multitude of techniques available to those skilled in the art for production and manipulation of various immunoglobulin molecules can be used in the present invention. Antibodies may be produced by a variety of means well known to those of skill in the art.

The production of non-human monoclonal antibodies, e.g., murine, lagomorpha, equine, etc., is well known and may be accomplished by, for example, immunizing the animal with a preparation containing the substrates of the invention. Antibody-producing cells obtained from the immunized animals are immortalized and screened, or screened first for the production of the desired antibody and then immortalized. For a discussion of general procedures of monoclonal antibody production see Harlow and Lane, Antibodies, A Laboratory Manual Cold Spring Harbor Publications, N.Y. (1988).

### EXAMPLES

### Example 1

### Synthesis of Lactosyl Ceramide and GM₃

Lactosyl Ceramide (d18:2) (7). Lactosyl sphingosine (2.2 g) was dissolved in 110 mL of a solution containing chloroform-methanol-40 mM phosphate buffer (pH=7.2) (60/40/9). The N-hydroxysuccinimide stearate (13.2 g) suspended in chloroform (55 mL) and triethylamine (1.1 mL) were then added and the reaction stirred at room temperature overnight. The solution was concentrated to dryness and the residue resuspended in acetone (110 mL). A methanolic solution of 10% magnesium chloride (11 mL) was then added and the solution cooled with dry ice for 1 hour. The precipitate was filtered and washed with cold acetone yielding 3.2 g of lactosyl ceramide (7) as a white solid. HPLC (Metachem Inertsil C8 column; 85% acetonitrile/15% water, UV 205 nm), Rₜ=23.1 min. See, Scheme 3. Additional variations in the protocol to synthesize lactosyl ceramide are shown in Table 2.

TABLE 2
Synthesis of Lactosyl Ceramide

| Amount of | | | | |
|---|---|---|---|---|
| compound 7 | | Solvent | Reaction Time | Lactosyl ceramide |
| 3 | mg | CH₃OH/Na₂HPO₄ | 4 hours | TLC |
| | | (1:1) | | |
| 68 | mg | CH₃OH/Na₂HPO₄ | 2 hours | TLC |
| | | (1:1) | | |
| 68 | mg | CHCl₃/CH₃OH/ | Overnight | TLC |
| | | Na₂HPO₄ (60:40:9) | | |
| 363 | mg | CHCl₃/CH₃OH/ | Overnight | TLC |
| | | Na₂HPO₄ (3:2:1) | | |
| 2.2 | g | CHCl₃/CH₃OH/ | Overnight | 3.2 g |
| | | Na₂HPO₄ (60:40:9) | | |

GM₃ (d18:2) (4). Lactosyl ceramide (7) (5.12 g) was suspended in water (4.1 L) and 3′-sialyllactose (253 g) and Zwittergent 3-14 (9.4 g). The pH was adjusted to 7.0, trans-sialidase (174 mL of cell homogenate) added and the reaction stirred for 2 hours. A Folch extraction was used to purify the GM₃ as follows. The KCl (64 g) was added to the reaction mixture and extracted with 29 L of CHCl₃/CH₃OH (2/1). The organic layer was separated and washed with water (19 L). The aqueous layer was extracted with 10 L of CHCl₃/CH₃OH (2/1) and the combined organic layers concentrated to dryness to afford 6.1 g of GM₃ (4). HPLC (MetaCapsil AMINO column; 85% acetonitrile, 15% 5 mM sodium phosphate buffer, pH=5.6; UV 197 nm), Rₜ=14.3 min.

As an alternative purification procedure following the enzyme reaction, the reaction mixture is concentrated ten fold using a 1 OK hollow fiber membrane purification system. Water (4 L) is then added and the solution diafiltered to a final volume of ˜0.4 L. Methanol (4 L) is then added to the retentate and the solution diafiltered, allowing the GM₃ to pass in the permeate. Concentration of the methanolic solution affords the GM₃.

Additional variations in the protocol to synthesize GM₃ are shown in Table 3.

TABLE 3
Optimization of GM₃ synthesis

| | | | Amt. of | | | |
|---|---|---|---|---|---|---|
| | | Molar | trans-sialidase | | | |
| Amount of | | excess of 3′- | cell lysate | | | |
| Lactosyl | | sialyllactose | vs. reaction | | | |
| Ceramide | | vs. 7 | volume | Temp. | Time | GM₃ |
| 8.8 | mg | 24x | 3% | RT | 6 h, 24 h | TLC |
| 2.2 | mg | 24x | 3% | RT | 6 h, 24 h | TLC |
| 8.8 | mg | 12x | 3% | RT | 6 h, 24 h | TLC |
| 8.8 | mg | 6x | 3% | RT | 6 h, 24 h | TLC |
| 8.8 | mg | 24x | 1.5% | RT | 6 h, 24 h | TLC |
| 8.8 | mg | 24x | 6% | RT | 6 h, 24 h | TLC |
| 220 | μg | 24x | 3% | 37° C. | overnight | TLC |
| 50 | μg | 96x | 3% | 37° C. | overnight | TLC |
| 50 | μg | 96x | 3% | RT | overnight | TLC |
| 200 | μg | 96x | 12% | 37° C. | overnight | TLC |
| 80 | mg | 100x | 3% | RT | 1 h | 37 mg |
| 80 | mg | 100x | 3% | RT | 16 h | 30 mg |
| 1.27 | g | 24x | 3% | 37° C. | 1 h | 280 mg |
| 652 | mg | 100x | 3% | RT | 1 h | 267 mg |

### Example 2

### Synthesis of GM₃, GM₂, and GM₁

Lactosyl Sphingadienine (d18:2) (2). (See, Scheme 2) The glucosyl sphingadienine (d18:2) (1) (0.50 mM, 6.8 g), HEPES (20 mM, 141 g), MnSO₄ (50 mM, 2.5 gm), UDP-galactose (4.0 mM, 76.7 g), NaN₃ (160 mM, 5.92 g) and water (30 L) were added to the reactor. The pH of the solution was adjusted to 7.4 and was maintained between 7.0-7.5. The β1,4-galactosyltransferase (900 units) was then added to the reaction mixture and the solution stirred for 12 hours yielding 7.1 gm of lactosyl sphingadienine (d18:2) (2) as determined by HPLC analysis. TLC (silica gel; CHCl₃/CH₃OH/H₂O/2.5 M NH₄OH-60/40/5/3), R_{f}=0.67; HPLC (YMC basic column; acetonitrile/sodium phosphate buffer (10 mM, pH 6.5); gradient of 30% to 80% acetonitrile; UV 205 nm), Rₜ=11.13 min and 11.48 min. MS (electrospray), m/z 620.2 [M−H]⁻.

Lyso-GM₃ (d18:2) (3). The 3′-sialyllactose (16 mM, 388.8 g) and Zwittergent (61 mM, 22.5 g) were added to the above reaction mixture and the reaction volume adjusted to 45 L with water. The suspension was warmed to 37° C. and the trans-sialidase (90,000 units) was added. The pH of the reaction mixture was maintained between 7.0-7.5 during the process. After 30 min., the solution was heated to 50° C. and then allowed to cool to room temperature. The reaction mixture was then concentrated to ˜5 L using a 10 K hollow fiber filtration unit. Water (10 L) was added to the retentate and the retentate concentrated to ˜5 L. The retentate was then diafiltered using 50% methanol in water (45 L) to maintain the retentate volume. Once the entire 50% methanol in water was consumed, methanol (10 L) was added to the retentate and concentrated to ˜2 L volume. The permeate collected during the 50% methanol/water filtration step, was then loaded directly onto a reversed phase (C18) chromatography column. The column was eluted first with 50% methanol in water, then with 85% methanol in water and the appropriate fractions containing the lyso-GM₃ (3) were collected yielding 8 gm of product as determined by HPLC. HPLC (YMC basic column; acetonitrile/sodium phosphate buffer (10 mM, pH 6.5) with a gradient of 30% to 80% acetonitrile; UV 205 nm), Rₜ=10.23 min and 10.56 min. MS (electrospray); m/z 911.3 ([M+H]⁻, calc=911.5).

GM₃ (d18:2) (4). The above column fractions containing the lyso-GM₃ were then concentrated to ˜1.5 L and THF (4.5 L) added. The solution was then cooled 10° C. and stearoyl chloride (165 mmoles, 50.0 gm) was added drop wise to the reaction solution with stirring while maintaining the pH at ˜7.7 by simultaneous addition of sodium hydroxide. After addition of the acid chloride was complete, the reaction mixture was stirred for 2 h and was filtered through a 1 μm bag filter. The filtrate was loaded onto a reversed phase (C18) column and washed with 50% methanol in water and 23% THF in water. The product is eluted first with 85% methanol in water and then with 90% methanol in water. Appropriate fractions were collected and evaporated to dryness. The residue is then purified using silica gel chromatography (CHCl₃, CH₃OH, water, concentrated NH₄OH; 50/40/2/0.1) to afford after concentration 8.7 gm of (4) as a white solid. TLC (silica gel; CHCl₃/CH₃OH/H₂O/2.5 N NH₄OH-60/40/5/3), R_{f}=0.60. HPLC (YMC basic column; acetonitrile/sodium phosphate buffer (1 mM, pH 6.85); gradient of 60% to 95% acetonitrile in 8 min; UV 205 run, at 1.4 mL/min), Rₜ=7.72 min. MS (electrospray); m/z 1177.7 ([M+H]⁻, calc=1177.7).

GM₂ (d18:2) (5). The GM₃ (7.1 mmoles, 8.4 g), Zwittergent (29.4 mmoles, 10.7 g), aqueous UDP-GalNAc/UDP-GlcNAc (14.7 mmoles), sodium azide (37 mmoles, 1.4 g) and GM₂ synthetase (28 units) were added to the reaction vessel and water was added to bring the volume to −7.0 L. The reaction mixture was heated at 37° C. for 12 hours. The reaction mixture was then concentrated to ˜0.7 L using a 10 K hollow fiber filtration unit. The retentate was diafiltered with water (7 L) to maintain the retentate volume. When the water was consumed, the retentate was then diafiltered with 100% methanol (7 L) while maintaining the retentate volume. During the methanol diafiltration, the product was collected in the permeate. The permeate was passed over an ion exchange column (Dowex 50, hydrogen form) and the appropriate fractions collected. The pH of the eluant was adjusted to 7.4 with sodium hydroxide and the solution loaded onto a reversed phase (C18) chromatography column. The column was washed with methanol/water (50/50, 80/20 and 90/10). Appropriate fractions were collected and concentrated to dryness. The residue was dissolved in water and freeze-dried to yield 7.6 g of GM₂ (5). HPLC (YMC basic column; 4.6×100 mm, 3 μm particle size; acetonitrile/sodium phosphate buffer (1 mM, pH 6.85) gradient, 60% to 95% acetonitrile in 8 min; UV 205 nm, at 1.4 mL/min), Rₜ=6.22 min, (d18:2,C18:0 GM₂). MS (electrospray), m/z 1380.8 ([M−H]⁻, calc=1380.8).

GM₁ (d 18:2) (6) (See, Scheme 2) is synthesized from GM₂ (d18:2) (5) by addition of galactose using β1,3-galactosyl transferase.

### Example 3

### Synthesis of GD₃ (See, Scheme 9, Top Reaction GM₃ (22)+Sialyltransferase and Sia Donor, Yields GD₃ (35))

GD₃ (d18:1) (35). Zwittergent (0.05 mg; 0.1%) was added to a methanolic solution of GM₃ (d18:1) (500 μM; 0.032 mg) and the solution evaporated with a stream of N₂ gas. —HEPES (50 mM, pH 7.0), CMP-sialic acid (0.02 mg), 10% cell lysate containing α-2,8-sialyltransferase-CST-68 (5 μL), MgCl₂ (10 mM; 0-1 mg), and water to a final reaction volume of 50 μL were then added. The reaction is incubated at 37° C. and for 3 hours. The sialylated products were purified using a Waters C18 Sep-pak light cartridge. The eluant was evaporated to dryness providing a mixture of GD₃, GT₃ and other multisialylated forms of GM₃. The percent conversion as calculated by HPLC as area %: GM₃, 39%; GD₃ 38%; GT₃, 15%; GQ₃, 7%. HPLC-MS (YMC basic column-4.6×100 mm; eluted with a gradient of 1 mM aqueous NH₄OH and acetonitrile from 50 to 95% MeCN over 8 min at 0.265 mUmin; UV=205 nn), GM₃ (ret time=29.54 min, m/z 1177.6 [M−H]⁻, calc=1177.7), GD₃ (ret time=22.34 min, m/z 1468.4 [M−H]⁻ calc=1468.8, m/z 733.9 [M−2H]²⁻, calc=733.9), GT₃ (ret time=18.70 min, m/z 1759.4 [M−H]⁻, calc=1759.9, m/z 879.4 [M−2H]²⁻, calc=879.5), and GQ₃ (ret time=17.19 min, m/z 1025.0 (M−2H]²⁻, calc=1025.0).

### Example 4

### Synthesis of Lyso-GD₃ (See, Scheme 6)

Lyso-GD₃ (d18:1) (8). Zwittergent (0.05 mg; 0.1%) was added to a methanolic solution of lyso-GM₃ (d18:1) (500 μM; 0.023 mg) and the solution evaporated with a stream of N₂ gas. HEPES (50 mM, pH 7.0), CMP-sialic acid (0.02 mg), 10% cell lysate containing α-2,8-sialyltransferase-CST-68 (5 μL), MgCl₂ (10 mM; 0.1 mg), and water to a final reaction volume of 50 μL were then added. The reaction was incubated at 37° C. for 3 hours. The sialylated products were purified using a Waters C18 Sep-pak light cartridge. The eluant was evaporated to dryness providing a mixture of lyso-GD₃, lyso-GT₃ and other multi-sialylated forms of lyso-GM₃. The percent conversion as calculated by HPLC as area %: lyso-GM₃, 39%; lyso-GD₃ 42%; lyso-GT₃, 16%; lyso-GQ₃, 3%. HPLC (YMC basic column-4.6×100 mm; eluted with a gradient of 10 mM aqueous sodium phosphate pH 6.5 and acetonitrile from 30 to 80% MeCN over 15 min at 1.0 mL/min; UV=205 nm), lyso-GM₃ (ret time=11 min), lyso-GD₃ (ret time=10 min), lyso-GT₃ (ret time=9 min), and Iyso-GQ₃ (ret time=9 min). The lyso-GD₃ (d18:1) (8) was purified from the mixture by reversed phase (C18) chromatography using a methanol/water gradient.

### Example 5

### Synthesis of Lyso-GD2 (See, Scheme 6)

Lyso-GD₂ (d18:1) (31). Zwittergent (0.075 mg; 0.1%) was added to a methanolic solution of lyso-GD₃ (d18:1) (1 mM; 0.060 mg) and the solution evaporated with a stream of N₂ gas. Sodium phosphate buffer (50 mM, pH 76.8), UDP-GalNAc (0.07 mg), 60% cell lysate containing GM₂ synthetase (30 μL), MnSO₄ (10 mM; 0.08 mg), and water to a final reaction volume of 50 μL are then added. The reaction was incubated at 37° C. for 72 hours. The product was then purified using a 10 K MWCO spinfilter, the permeate discarded and methanol added to the retentate. Centrifugation at 10,000 rpm eluted the product in the permeate. The eluant was evaporated to dryness and contained a mixture of lyso-GD₃ and lyso-GD₂. The percent conversion as calculated by HPLC as area %: lyso-GD₂, 38%; lyso-GD₃, 61 %. HPLC-MS (YMC basic column; 2×100 mm; eluted with a gradient of 1 mM aqueous NH₄OH and acetonitrile from 30 to 100% ACN over 25 min at 0.250 mL/min; UV=205 nm), lyso-GD₃ (UV ret time=14.383 min, m/z 1205.5 [M−H]⁻, calc=1204.5), lyso-GD₂ (UV ret time=14.0 min, m/z 1408.4 [M−H]⁻ calc=1407.4).

### Example 6

### Synthesis of Lyso-GM3 (See, Scheme 5)

Lyso-GM₃ (d18:1) (18). 3′-sialyllactose (16 mM, 444.5 g), Zwittergent 3-14 (0.05%, 20.1 g), and lactosyl sphingosine (17; 0.4 mM, 10.01 g) was added to 20 L USP water, in a temperature controlled reactor. The solution was heated to 37° C. The remaining 19.25 L USP water and the α2-3 trans-sialidase (2000 Units/L, 0.95 L) were added to the reactor, bringing the total synthesis volume to 40.2 L. The pH was adjusted to 7.0 and the mixture was allowed to stir for 30 min at 37° C. The solution was then heated to 50° C. for an additional 30 min and the reaction mixture then cooled to room temperature.

The reaction mixture (40.2 L) was then concentrated to one eighth of its original volume (5 L) using a 10 K hollow fiber membrane purification system. Water (10 L) was then added to the retentate, and the retentate diafiltered with an additional 40 L of water. The retentate was then concentrated to 5 L volume and 10 L of methanol/water (50/50) was added to the retentate. The retentate was then diafiltered with 40 L of methanol/water (50/50) and the retentate concentrated to 5 L volume. The lyso-GM₃ (18) eluted in the permeate at this step.

The permeate (methanol/water 50/50) containing the lyso-GM₃ (51 L) was then loaded onto a reversed phase (C18) chromatography column. The column was washed with 10 column volumes (5 L) of methanol:water (50/50) and the product eluted with 10 column volumes (5 L) of methanol:water (85/15). Appropriate fractions were collected and concentrated to dryness by rotoevaporation yielding 12.03 g of lyso-GM₃ (18). HPLC (YMC basic column, 4.6×100 mm; gradient, 30% to 80% acetonitrile/10 mM NaH₂PO₄-pH 6.5; 1.0 mL/min over 15 min.; UV=205), Rₜ=11.1 min.

TABLE 4
Trans-sialidase Reaction, lactosyl sphingosine.

| Amount of | | | |
|---|---|---|---|
| lactosyl | | Amount of trans- | Conversion Yield |
| sphingosine (17) | | sialidase | (determined by HPLC) |
| 2.5 | mg | 4250 U/L | 96% |
| 2.5 | mg | 8500 U/L | 94% |
| 2.5 | mg | 17000 U/L | 92% |
| 2.5 | mg | 889 U/L | 96% |
| 2.5 | mg | 444 U/L | 89% |
| 2.5 | mg | 222 U/L | 77% |
| 2.5 | mg | 111 U/L | 63% |
| 124 | mg | 1000 U/L, + 2496 U/L | 95% |
| 1 | g | 4000 U/L, + 2242 U/L | 90% |
| 373 | mg | 4000 U/L | 92% |
| 10 | g | 2000 U/L | 94% |
| 50 | g | 2000 U/L | 96% |

TABLE 5
Membrane Purification of lyso-GM₃ (18).

| | Concentration | | CH₃OH/H₂O | CH₃OH/H₂O | 100% |
|---|---|---|---|---|---|
| Hollow Fiber | (from original | H₂O¹ | (50/50)¹ | (80/20)¹ | CH₃OH¹ |
| Membrane | reaction | (diafiltration | (diafiltration | (diafiltration | (diafiltration |
| (size) | volume) | amount)² | amount)² | amount)² | amount)² |
| 10 K | 5 fold | 5 volumes | 5 volumes³ | NT | 2 volumes |
| 10 K | 10 fold | 10 volumes | 10 volumes³ | NT | 5 volumes |
| 10 K | 8 fold | 10 volumes | 10 volumes³ | NT | 2 volumes |
| 3 K | 10 fold | 10 volumes | 10 volumes³ | 5 volumes³ | NT |

¹The retentate was diluted with this solvent to the original volume of the reaction mixture.
²Amount of solvent used to diafiltrate the retentate at this step. After diafiltration, the

retentate was concentrated again. ³The lyso-GM₃ (d18:1) began to elute at this solvent concentration.

### Example 7

### Synthesis of Lyso-GM2 (See, Scheme 5)

Lyso-GM₂ (d18:1) (19). The lyso-GM₃ (18; 1 mM, 10.04 g), Zwittergent 3-14 (0.15%, 16.5 g), manganese sulfate (10 mM, 18.60 g), sodium azide (0.02%, 2.2 g), and UDP-GalNAc (4 mM, 4.29 L) were added to 1.5 L USP water in a temperature and pH controlled reactor. The reaction mixture was heated to 37EC and the pH was adjusted to 7. The GM₂-Synthetase (GalNAc transferase, 7.6 U/L, 0.85 L) and the remaining 4.36 L USP water was then added to the reactor, bringing the final volume to 11 L. The reaction mixture stirred for 65 h at 37° C. with pH control. The solution was then brought to 50° C., heated for an additional 30 min. and then was cooled to room temperature.

The reaction mixture (11 L) was then concentrated to a quarter of its original volume (4 L) using a 3 K hollow fiber membrane purification system. Water (10 L) was then added to the retentate and the retentate diafiltered with an additional 10 L of water. The retentate was then concentrated to 5 L volume and 10 L of methanol/water (25/75) was added to the retentate. The retentate was diafiltrated with an additional 40 L of methanol/water (25/75) and was then concentrated to 5 L volume. Methanol/water (35/65) (10 L) was then added to the retentate, which was diafiltrated with an additional 40 L methanol/water (35/65) and then concentrated to 5 L volume. Methanol/water (50/50) (10 L) was added to the retentate, which was diafiltrated with an additional 40 L methanol/water (50/50) and then concentrated to 5 L volume. The lyso-GM₂ (19) was found to elute primarily in the first two methanol/water eluants which were combined and loaded onto a reverse phase (C18) chromatography column. The column was washed with 10 column volumes (5 L) of methanol/water (50/50). The product was eluted from the column with 10 column volumes (5 L) of methanol/water (75/25) and 10 column volumes (5 L) of methanol/water (80/20). Appropriate fractions were collected and concentrated to dryness.

The residue was dissolved in 90 mL of CH₃CN/CH₃OH/CH₂CI₂ (1/1/1) and divided into four portions of equal volume. Each sample was loaded onto a silica gel chromatography equilibrated in CH₃CN/CH₃OH/CH₂Cl₂/NH₄OH (30/30/30/5). The column was washed with eight column volumes of CH₃CN/CH₃OH/CH₂Cl₂/NH₄OH (30/30/30/5) and the product eluted with CH₃CN/CH₃/NH₄OH (20/50/10). Appropriate fractions were collected and concentrated to dryness by rotoevaporation yielding a total of 9.66 g of lyso-GM₂ (19). HPLC (YMC basic column, 4.6×100 mm; gradient, 30% to 80% acetonitrile/10 mM NaH₂PO₄-pH 6.5; 1.0 mL/min over 15 min.; UV=205), Rₜ=10.78 min.

TABLE 6
GM2-Synthetase(GalNAc transferase)
Reaction, lyso-GM₃ (d18:1).

| | | UDP- | | | |
|---|---|---|---|---|---|
| Amount of | | GalNAc | Amount of | | |
| Lyso-GM₃ | | Con- | GM₂- | % Conversion | |
| (d18:1) | | centration | Synthetase | (determined by HPLC) | |
| 0.9 | mg | 2 mM | 4 U/L | | 86% in 48 h |
| 0.9 | mg | 4 mM | 4 U/L | | 91% in 48 h |
| 9 mg, 0.9 mg | | 2 mM | 8 U/L | 87% in 24 h | 95% in 48 h |
| 9 | mg | 4 mM | 8 U/L | 96% in 24 h | |
| 9 | mg | 2 mM | 12 U/L | 95% in 24 h | |
| 183 | mg | 4 mM | 8 U/L | 91% in 24 h | 97% in 42 h |
| 502 | mg | 4 mM | 8 U/L | 91% in 24 h | 97% in 42 h |
| 10 | g | 4 mM | 7.6 U/L | | 100% in 65.5 h |
| 4.93 | g | 4 mM | 7.6 U/L | | 98% in 65 h |

### Example 8

### Synthesis of Lyso-GM₁ (See, Scheme 5)

Lyso-GM₁ (d18:1) (20). Lyso-GM₂ (19; 0.8 mM, 5.00 g), UDP-Gal (1.4 mM, 5.05 g), manganese chloride (10 mM, 11.08 g), and sodium azide (0.02%,1.12 g) was added to 3 L of water, in a 6 L flask. The flasks contents were heated to 37EC and placed in a 37EC incubator. The remaining 2.21 L of water and GM₁-Synthetase (β1-3 Galactosyl Transferase, 7% crude lysate, 0.39 L) was added to the flask, bringing the final volume to 5.6 L. The reaction mixture was stirred and the pH controlled to remain around pH 6.5, overnight for 16 h at 37° C. The solution was then brought to 50° C., heated for an additional 30 min. and then was cooled to room temperature.

The reaction mixture (5.6 L) was then concentrated to a third of its original volume (2 L) using a 3 K hollow fiber membrane purification system. Water (1 L) was added to the retentate and the retentate diafiltered with an additional 9 L of water. The retentate was then concentrated to 2 L volume and methanol/water (50/50) (1 L) was then added to the retentate. The retentate was then diafiltrated with an additional 19 L methanol/water (50/50) and concentrated to 2 L volume. The lyso-GM₁ (20) eluted in the methanol/water (50/50) permeate.

The permeate (50/50) (20 L) containing the product was then loaded onto a reversed phase (C18) chromatography column. The column was washed with 10 column volumes (5 L) of methanol/water (50/50). The product was eluted with 10 column volumes (5 L) of methanol/water (90/10). Appropriate fractions were collected and concentrated to afford 4.8 g of lyso-GM₁. HPLC (YMC basic column, 4.6×100 mm; 53% acetonitrile/47% 10 mM NaH₂PO₄-pH 6.5; 1.0 mL/min over 7 min.; UV=205), Rₜ=5.03 min. ¹H NMR (500 MHz, CD₃OD) δ 5.84 (m, 1H, vinyl proton), 5.50 (m, 1H, vinyl proton), 4.44 (d, J 8.0 Hz, 1H), 4.40 (d, J 8.0 Hz, 1H), 4.30 (m, 1H), 4.10-4.20 (m, 1H), 3.20-3.40 (m, sugar ring protons), 2.75 (dd, J 4.5 and 12.5 Hz, 1H), 2.10 (q, 3H), 2.01 (2s, 6H, 2Ac), 1.42 (t, 3H), 1.30 (s, 22H), 0.90 (t, 3H, CH₃).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. An in vitro, cell-free, enzymatic method for preparing a compound having the formula: 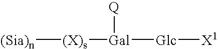
in which
X¹ is a member selected from substituted or unsubstituted alkyl, a detectable label and a targeting moiety;
X is a member selected from: 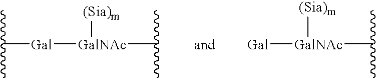
wherein
m is an integer from 0 to 20;
Q is a member selected from: 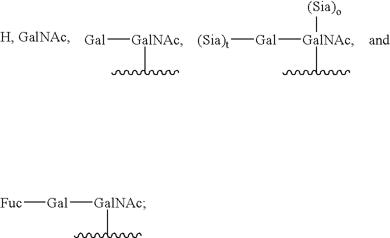
n, o and s are integers independently selected from 0 to 20;
said method comprising:
(a) contacting with a trans-sialidase and a Sia donor, a substrate having the structure: 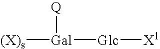
under conditions appropriate for said trans-sialidase to transfer a Sia moiety from said donor to said substrate, thereby forming said compound.

2. The method according to claim 1, further comprising:
(b) contacting the compound formed in step (a) with a GalNAc-transferase and a GalNAC donor under conditions appropriate for said GalNAc-transferase to transfer a GalNAc moiety from said donor to said compound formed in step (a).

3. The method according to claim 2, further comprising:
(c) contacting the compound formed in step (b) with a Gal-transferase and a Gal donor under conditions appropriate for said Gal-transferase to transfer a Gal moiety from said donor to said compound formed in step (b).

4. The method according to claim 3, further comprising:
(d) contacting the compound formed in step (c) with a trans-sialidase and a Sia donor under conditions appropriate for said trans-sialidase to transfer a Sia moiety from said donor to said compound formed in step (c).

5. The method according to claim 4, further comprising:
(e) contacting the compound formed in step (d) with a Sia-transferase and a Sia donor under conditions appropriate for said Sia-transferase to transfer a Sia moiety from said donor to said compound formed in step (d).

6. The method according to claim 3, further comprising:
(d) contacting the compound formed in step (c) with a Fuc-transferase and a Fuc donor under conditions appropriate for said Fuc-transferase to transfer a Fuc moiety from said donor to said compound formed in step (c).

7. The method of claim 1, further comprising:
(b) contacting the compound formed in step (a) with a Sia-transferase and a Sia donor under conditions appropriate for said Sia-transferase to transfer a Sia moiety from said donor to said compound formed in step (a).

8. The method of claim 7, further comprising:
(c) contacting the compound formed in step (b) with a GalNAc-transferase and a GalNAc donor under conditions appropriate for said GalNAc-transferase to transfer a GalNAc moiety from said donor to said compound formed in step (b).

9. The method of claim 8, further comprising:
(d) contacting the compound formed in step (c) with a Gal-transferase and a Gal donor under conditions appropriate for said Gal-transferase to transfer a Gal moiety from said donor to said compound formed in step (c).

10. The method of claim 9, further comprising:
(e) contacting the compound formed in step (d) with a trans-sialidase and a Sia donor under conditions appropriate for said trans-sialidase transfer a Sia moiety from said donor to said compound formed in step (d).

11. The method of claim 10, further comprising:
(f) contacting the compound formed in step (e) with a Sia-transferase and a Sia donor under conditions appropriate for said Sia-transferase to transfer a Sia moiety from said donor to said compound formed in step (e).

12. The method of claim 7, further comprising:
(c) contacting the compound formed in step (b) with a Sia-transferase and a Sia donor under conditions appropriate for said Sia-transferase to transfer a Sia moiety from said donor to said compound formed in step (b).

13. The method of claim 12, further comprising:
(d) contacting the compound formed in step (c) with a GalNAc-transferase and a GalNAc donor under conditions appropriate for said GalNAc-transferase to transfer a GalNAc moiety from said donor to said compound formed in step (c).

14. The method of claim 13, further comprising:
(e) contacting the compound formed in step (d) with a Gal-transferase and a Gal donor under conditions appropriate for said Gal-transferase to transfer a Gal moiety from said donor to said compound formed in step (d).

15. The method of claim 14, further comprising:
(f) contacting the compound formed in step (e) with a trans-sialidase and a Sia donor under conditions appropriate for said trans-sialidase to transfer a Sia moiety from said donor to said compound formed in step (e).

16. The method of claim 1, further comprising:
(g) prior to step (a), contacting a substrate having the formula:
Q-Glc-X¹
with a Gal-transferase and a Gal donor under conditions appropriate for said Gal-transferase to transfer a Gal moiety from said donor to said substrate

17. The method of claim 1, further comprising:
(g) prior to step (a), contacting a substrate having the formula:
Q-Gal-Glc-X¹
with a GalNAc-transferase and a GalNAc donor under conditions appropriate for said GalNAc-transferase to transfer a GalNAc moiety from said donor to said substrate.

18. The method according claim 17, further comprising:
(h) contacting the compound formed in step (g) with a Gal-transferase and a Gal donor under conditions appropriate for said Gal-transferase to transfer a Gal moiety from said donor to said compound formed in step (g).

19. The method of claim 18, further comprising:
(i) following step (a), contacting the compound formed in step (a) with a Sia-transferase and a Sia donor under conditions appropriate for said Sia-transferase to transfer a Sia moiety from said donor to said compound formed in step (a).

20. The method of claim 19, further comprising:
(j) repeating step (i) a selected number of times, thereby forming a poly(sialic acid) substituent on said compound.

21. The method of claim 1, further comprising:
(k) contacting the compound formed in step (a) with a Sia-transferase and a Sia donor under conditions appropriate for said Sia-transferase to transfer a Sia moiety from said donor to said compound formed in step (a).

22. The method of claim 21, further comprising:
(l) repeating step (k) a selected number of times, thereby forming a poly(sialic acid) substituent on said compound.

23. The method of claim 1, wherein X¹ is: 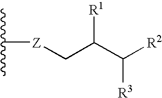
in which
Z is selected from O, S and NR⁵;
R¹ and R² are members independently selected from NHR⁴, SR⁴, OR⁴, OCOR⁴, OC(O)NHR⁴, NHC(O)OR⁴, OS(O)₂OR⁴, C(O)R⁴, NHC(O)R⁴, detectable labels, and targeting moieties
in which
R⁴ and R⁵ are members independently selected from H, substituted or unsubstituted alkyl, substituted or unsubstituted heteroalkyl, detectable labels and targeting moieties; and
R³ is substituted or unsubstituted alkyl and substituted or unsubstituted heteroalkyl groups.

24. The method according to claim 23, wherein R¹ is a member selected from NH₂, OH and SH, said method further comprising acylating R¹.

25. The method of claim 23, wherein X¹ is: 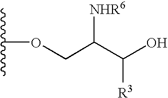
wherein
R⁶ is a member selected from H, C(O)R⁷, detectable labels, and targeting moieties
in which
R⁷ is a member selected from substituted or unsubstituted alkyl, substituted or unsubstituted heteroalkyl, detectable labels and targeting moieties.

26. The method according to claim 25, wherein said compound is a member selected from GM₂, GM₁, GD₁ₐ. GT₁ₐ, Fuc-GM₁, GD₃, GD₂, GD_{1b}, GT_{1b}, GQ_{1b}, GM_{1b}, GD_{1α}, GT_{1β}, GQ_{1B}, GT₃, GT₂, GT_{1c}, GQ_{1c}, globosindes, and polysialylated lactose.

27. A compound having the formula: 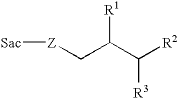
in which
Z is selected from O, S and NR₅;
R¹ and R² are members independently selected from NHR⁴, SR⁴, OR⁴, OCOR⁴, OC(O)NHR⁴, NHC(O)OR⁴, OS(O)₂OR⁴, C(O)R⁴, NHC(O)R⁴, detectable labels, and targeting moieties
in which
R⁴ and R⁵ are members independently selected from H, substituted or unsubstituted alkyl, substituted or unsubstituted heteroalkyl, detectable labels and targeting moieties;
Sac is a member selected from mono- and oligo-saccharide; and
R³ is substituted or unsubstituted alkyl having at least two degrees of unsaturation and substituted or unsubstituted heteroalkyl groups.

28. The compound according to claim 27 wherein R³ is unsubstituted alkyl having two double bonds, and Sac is other than glucosyl.

29. The compound according to claim 27, wherein R³ includes at least one triple bond.

30. The compound according to claim 27, wherein said compound is a member selected from d18:2 and d18:1:1.

31. A pharmaceutical formulation comprising a compound according to claim 27 in admixture with a pharmaceutically acceptable excipient.
